(19) United States
(12) Patent Application Publication
(10) Pub. No.: US 2024/0254495 A1
(43) Pub. Date: Aug. 1, 2024

(54) Therapeutic Engineered Microbial Cell Systems and Methods for Treating Conditions in Which Oxalate is Detrimental

(71) Applicant: Unlocked Labs Inc., Laramie, WY (US)

(72) Inventor: Christoph Geisler, Laramie, WY (US)

(21) Appl. No.: 18/688,938

(22) PCT Filed: Sep. 8, 2022
(86) PCT No.: PCT/US2022/076108

§ 371 Date: Mar. 4, 2024

Related U.S. Application Data
(60) Provisional application No. 63/261,034, filed on Sep. 9, 2021.

(51) Int. Cl. C12N 15/75; A61K 35/742; C12N 1/20; C12N 9/88

(52) U.S. Cl. CPC C12N 15/75; A61K 35/742; C12N 1/20; C12N 9/88; C12Y 401/01002

(57) ABSTRACT

The present disclosure relates to engineered microbial cells that have been engineered to comprise one or more oxalate catabolismgenes that are expressed under the control of non-native, non-inducible promoters. Thus, the genetically engineered microbial cells and pharmaceutical compositions comprising the microbial cells are useful in degrading oxalate inside or outside the engineered microbial cell, resulting in a reduction of the concentration of oxalate outside the cell. The engineered microbial cells of the present disclosure are useful in methods of treating or preventing diseases associated with disorders in which oxalate is detrimental, such as hyperoxalurias. The engineered microbial cells of the present disclosure are also useful in methods of treating calcium oxalate nephrocalcinosis, calcium oxalate nephrolithiasis and calcium oxalate urolithiasis.

### CROSS REFERENCE TO RELATED APPLICATIONS

This application is a national phase entry under 35 U.S.C. § 371 of International Patent Application PCT/US2022/076108, filed Sep. 8, 2022, designating the United States of America and published in English as International Patent Publication WO2023/039469 on Mar. 16, 20239, which claims the benefit under Article 8 of the Patent Cooperation Treaty to United States Provisional Patent Application Ser. No. 63/261,034, filed Sep. 9, 2021, the entireties of which are hereby incorporated by reference.

### INCORPORATION BY REFERENCE

The ST.26 XML Sequence listing named “09796 US ST26 Sequence Listing”. created on Feb. 7, 2024, and having a size of 32.768 bytes, is hereby incorporated herein by this reference in its entirety.

### BACKGROUND

Oxalic acid is a dicarboxylic acid with the formula HO2C—CO2H. Oxalic acid is found primarily as the conjugate base oxalate in biological organisms. Oxalate is found in many foods, such as, e.g., spinach, rhubarb, strawberries, cranberries, nuts, cocoa, chocolate, peanut butter, sorghum, and tea. Oxalate is also a terminal metabolic product in humans and other mammals, and is excreted unchanged by the kidneys into the urine. When combined with calcium, oxalic acid produces an insoluble product, calcium oxalate, which is the most prevalent chemical compound found in kidney stones.

Because mammals do not endogenously produce enzymes having the ability to degrade oxalate, oxalate levels in an individual are normally held in check by excretion and low absorption of dietary oxalate. Elevated concentrations of oxalate are associated with a variety of pathologies, such as primary hyperoxaluria, enteric hyperoxaluria, and idiopathic hyperoxaluria ((Hoppe & Blau, 2014) and (Witting et al., 2021). Increased oxalate levels can be caused by overconsumption of oxalate-rich foods, by hyperabsorption of oxalate from the intestinal tract, by excessive endogenous oxalate production, or by insufficient excretion of oxalate in the urine. Hyperabsorption of oxalate in the colon and small intestine can be associated with intestinal diseases, including hyperabsorption caused by diseases of bile acid and fat malabsorption; ileal resection; and, for example, by steatorrhea due to celiac disease, exocrine pancreatic insufficiency, intestinal disease, and liver disease.

Hyperoxaluria, or increased urinary oxalate excretion, is associated with a number of health problems related to the deposition of calcium oxalate stones in the kidney tissue (nephrocalcinosis) or urinary tract (e.g., kidney stones, urolithiasis, and nephrolithiasis). Calcium oxalate may also be deposited in, e.g., the eyes, blood vessels, joints, bones, muscles, heart and other major organs, causing damage to these organs. Sec, e.g., (Hoppe & Blau, 2014) and (Monico, Persson, Ford, Rumsby, & Milliner, 2002). The effects of increased oxalate levels can appear in various tissues. For example, deposits in small blood vessels can cause painful skin ulcers that do not heal, deposits in bone marrow cause anemia, deposits in bone tissue cause fractures or affect growth in children, and calcium oxalate deposits in the heart cause abnormalities of heart rhythm or poor heart function.

Existing methods to treat elevated oxalate levels are not always effective and intensive dialysis and organ transplantation may be required in many patients with primary hyperoxaluria. Existing therapies for various hyperoxalurias include high-dose pyridoxine, orthophosphate, magnesium, iron, aluminum, potassium citrate, cholestyramine, and glycosaminoglycan treatment, as well as regimes for adjusting diet and fluid intake, for dialysis, and for surgical intervention, such as renal and liver transplantation.

### SUMMARY OF THE INVENTION

The present invention provides engineered microbial cells, pharmaceutical compositions thereof, and methods of modulating and treating disorders in which oxalate is detrimental.

Specifically, the microbial cells disclosed herein have been engineered to comprise for example, one or more oxalate catabolismgenes. The one or more oxalate catabolismgenes may be exogenous to the microbial cell and/or the one or more oxalate catabolismgenes may be expressed under the control of non-native promoters. The engineered microbial cells can be probiotic cells. The engineered cells can be eukaryotic, e.g., fungal, e.g., Saccharomyces boulardii. The engineered cells can be bacterial, e.g., from the genus Lactobacillus or from the genus Bacillus or can be archaeal. In one embodiment, the engineered microorganism constitutively expresses the oxalate catabolismgene (s). In another embodiment, the microorganism is probiotic. These engineered microbial cells are safe and well tolerated and augment the innate activities of the subject's microbiome to achieve a therapeutic effect.

In some embodiments, the disclosure provides a microbial cell that has been genetically engineered to comprise one or more genes, gene cassettes, and/or synthetic circuits encoding one or more oxalate catabolismenzyme(s) or oxalate catabolismpathway, and is capable of metabolizing oxalate and/or other metabolites, such as oxalyl-CoA. Thus, the genetically engineered microbial cells and pharmaceutical compositions comprising the microbial cells may be used to treat and/or prevent diseases associated with disorders in which oxalate is detrimental, such as hyperoxalurias and kidney stones.

In some embodiments, the disclosure provides a microbial cell that has been engineered to comprise gene sequence(s) encoding one or more oxalate catabolismenzyme(s). In some embodiments, the disclosure provides a microbial cell that has been engineered to comprise gene sequence(s) encoding one or more oxalate catabolismenzyme(s) and is capable of reducing the level of oxalate and/or other metabolites, for example, oxalyl-CoA. In some embodiments, the microbial cell has been engineered to comprise gene sequence(s) encoding one or more extracellular oxalate decarboxylase enzymes. In some embodiments, the microbial cell has been engineered to comprise gene sequence(s) encoding one or more transporter(s) (importer(s)) of oxalate. In some embodiments, the microbial cell has been engineered to comprise gene sequence(s) encoding one or more transporter(s) (exporter(s)) of formate. In some embodiments, the engineered microbial cells comprise gene sequence(s) encoding one or more polypeptide(s) which mediate both the transport (import) of oxalate and the transport (export) of formate (e.g., oxalate:formate antiporter(s)). In some embodiments, the engineered microbial cells comprise gene sequence(s) encoding one or more of the following: (i) one or more transporter(s) of oxalate; (ii) one or more transporter(s) of formate; (iii) one or more polypeptide(s) which mediate both the transport (import) of oxalate and the export of formate (e.g., oxalate:formate antiporter(s)); and (iv) any combination thereof. In some embodiments, the microbial cell has been engineered to comprise gene sequence(s) encoding one or more oxalate catabolismenzyme(s) and one or more transporter(s) (importer(s)) of oxalate. In some embodiments, the microbial cell of the disclosure has been genetically engineered to comprise gene sequence(s) encoding one or more oxalate catabolismenzyme(s) and one or more transporter(s) of formate. In some embodiments, genetically engineered microbial cells comprise gene sequence(s) encoding one or more oxalate catabolismenzyme(s) and one or more polypeptide(s), which mediate both the transport (import) of oxalate and the export of formate (e.g., oxalate:formate antiporter(s)). In some embodiments, the microbial cell has been engineered to comprise gene sequence(s) encoding one or more oxalate catabolismenzyme(s) and gene sequence(s) encoding one or more of the following: (i) one or more transporter(s) of oxalate; (ii) one or more transporter(s) of formate; (iii) one or more polypeptide(s) which mediate both the transport (import) of oxalate and the export of formate (e.g., oxalate:formate antiporter(s)); and (iv) any combination thereof.

In some embodiments, the gene sequence(s) encoding one or more oxalate catabolism enzyme(s) is operably linked to a non-inducible promoter. In some embodiments, the gene sequence(s) encoding one or more oxalate transporter(s) (importer(s)) is operably linked to a non-inducible promoter. In some embodiments, the gene sequence(s) encoding one or more extracellular oxalate decarboxylase enzymes is operably linked to a non-inducible promoter. In some embodiments, the gene sequence(s) encoding one or more transporter(s) of formate is operably linked to a non-inducible promoter. In some embodiments, the gene sequence(s) encoding one or more polypeptide(s) which mediate both the transport (import) of oxalate and the export of formate (e.g., oxalate:formate antiporter(s)) is operably linked to a non-inducible promoter. In some embodiments, the gene sequence(s) encoding one or more oxalate catabolismenzyme(s) and the gene sequence(s) encoding one or more oxalate transporter(s) (importer(s)) are operably linked to a non-inducible promoter. In some embodiments, the gene sequence(s) encoding one or more oxalate catabolismenzyme(s) and the gene sequence(s) encoding one or more transporter(s) of formate are operably linked to a non-inducible promoter. In some embodiments, the gene sequence(s) encoding one or more oxalate catabolismenzyme(s) and the gene sequence(s) encoding one or more polypeptide(s) which mediate both the transport (import) of oxalate and the export of formate (e.g., oxalate:formate antiporter(s)) are operably linked to a non-inducible promoter. In some embodiments, any one or more of the following gene sequences, if present in the microbial cell, are operably linked to a non-inducible promoter: (i) gene sequence(s) encoding one or more oxalate catabolismenzyme(s); (ii) gene sequence(s) encoding one or more oxalate transporter(s); (iii) gene sequence(s) encoding one or more transporter(s) of formate; and (iv) gene sequence(s) encoding one or more polypeptide(s) which mediate both the transport (import) of oxalate and the export of formate (e.g., oxalate:formate antiporter(s)).

In some embodiments, the disclosure provides a microbial cell which has been engineered to comprise gene sequence(s) encoding one or more oxalate catabolismenzyme(s) operably linked to a non-inducible promoter, e.g. a constitutive promoter. In some embodiments, the disclosure provides a microbial cell which has been engineered to comprise gene sequence(s) encoding one or more oxalate transporter(s) (importer(s)) operably linked to a non-inducible promoter, e.g. a constitutive promoter. In some embodiments, the disclosure provides a microbial cell which has been engineered to comprise gene sequence(s) encoding one or more transporter(s) of formate operably linked to a non-inducible promoter, e.g. a constitutive promoter. In some embodiments, the disclosure provides a microbial cell which has been engineered to comprise gene sequence(s) encoding one or more polypeptide(s) which mediate both the transport (import) of oxalate and the export of formate (e.g., oxalate:formate antiporter(s)) operably linked to a non-inducible promoter, e.g. a constitutive promoter. In some embodiments, the gene sequence(s) encoding one or more oxalate catabolism enzyme(s) and the gene sequence(s) encoding one or more oxalate transporter(s) (importer(s)) are operably linked to a non-inducible promoter, e.g. a constitutive promoter. In some embodiments, the gene sequence(s) encoding one or more oxalate catabolismenzyme(s) and the gene sequence(s) encoding one or more transporter(s) of formate are operably linked to a non-inducible promoter, e.g. a constitutive promoter. In some embodiments, the gene sequence(s) encoding one or more oxalate catabolismenzyme(s) and the gene sequence(s) encoding one or more polypeptide(s) which mediate both the transport (import) of oxalate and the export of formate (e.g., oxalate:formate antiporter(s)) are operably linked to a non-inducible promoter, e.g. a constitutive promoter. In some embodiments, any one or more of the following gene sequences, if present in the microbial cell, are operably linked to a non-inducible promoter: (i) gene sequence(s) encoding one or more oxalate catabolismenzyme(s); (ii) gene sequence(s) encoding one or more oxalate transporter(s); (iii) gene sequence(s) encoding one or more transporter(s) of formate; and (iv) gene sequence(s) encoding one or more polypeptide(s) which mediate both the transport (import) of oxalate and the export of formate (e.g., oxalate:formate antiporter(s)).

In some embodiments, the invention provides a microbial cell that has been engineered to comprise gene sequence(s) encoding one or more oxalate catabolismenzyme(s) that is operably linked to a non-inducible, constitutive promoter.

In some embodiments, the disclosure provides a microbial cell that has been engineered to comprise gene sequence(s) encoding one or more polypeptide(s) capable of reducing the level of oxalate and/or other metabolites, for example, oxalyl-CoA, in low-oxygen environments, e.g., the digestive tract. In some embodiments, the microbial cell that has been engineered to comprise gene sequence(s) encoding one or more of the following: (i) one or more oxalate catabolismenzyme(s); (ii) one or more oxalate transporter(s); (ii) one or more formate transporter(s); and (iv) one or more oxalate:formate antiporter(s). In some embodiments, the microbial cell has been genetically engineered to comprise expression of one or more oxalate catabolismenzyme(s) and is capable of processing and reducing levels of oxalate, and/or oxalyl-CoA e.g., in low-oxygen environments, e.g., the digestive tract. Thus, in some embodiments, the genetically engineered microbial cells and pharmaceutical compositions comprising the microbial cells of the disclosure may be used to import excess oxalate and/or oxalyl-CoA into the microbial cell in order to treat and/or prevent conditions associated with disorders in which oxalate is detrimental, such as hyperoxalurias and nephrolithiasis. In some embodiments, the genetically engineered microbial cells and pharmaceutical compositions comprising the microbial cells of the disclosure may be used to catabolize excess oxalate outside the microbial cell in order to treat and/or prevent conditions associated with disorders in which oxalate is detrimental, such as hyperoxalurias and nephrolithiasis. In some embodiments, the genetically engineered microbial cells and pharmaceutical compositions comprising the microbial cells of the disclosure may be used to convert excess oxalate into CO₂ and formate in order to treat and/or prevent conditions associated with disorders in which oxalate is detrimental, such as hyperoxalurias and nephrolithiasis. In some embodiments, the genetically engineered microbial cells and pharmaceutical compositions comprising the microbial cells of the disclosure may be used to convert excess oxalate and/or oxalyl-CoA into non-toxic molecules in order to treat and/or prevent conditions associated with disorders in which oxalate is detrimental, such as hyperoxalurias and nephrolithiasis.

The present invention provides recombinant microbial cells, pharmaceutical compositions thereof, and methods of modulating and treating disorders in which oxalate is detrimental. The genetically engineered microbial cells and pharmaceutical compositions comprising the microbial cells of the invention may be used to convert excess oxalate and/or oxalic acid into non-toxic molecules in order to treat and/or prevent conditions associated with disorders in which oxalate is detrimental, such as hyperoxalurias and nephrolithiasis. In some embodiments, a microbial cell of the invention has been engineered to comprise at least one heterologous or endogenous gene encoding at least one oxalate catabolismenzyme and is capable of processing and reducing levels of oxalate, in low-oxygen environments, e.g., the digestive tract. In some embodiments, a microbial cell of the invention has been engineered to comprise at least one heterologous or endogenous gene encoding an importer of oxalate and is capable of reducing levels of oxalate, in low-oxygen environments, e.g., the digestive tract. In some embodiments, a microbial cell of the invention has been engineered to comprise at least one heterologous or endogenous gene encoding a transporter of formate and is capable of reducing levels of oxalate, in low-oxygen environments, e.g., the digestive tract. In some embodiments, a microbial cell of the invention has been engineered to comprise at least one heterologous or endogenous gene encoding an oxalate:formate antiporter and is capable of reducing levels of oxalate, in low-oxygen environments, e.g., the digestive tract.

In some embodiments, the at least one oxalate catabolismenzyme converts oxalate to formate or formyl CoA. In some embodiments, the at least one oxalate catabolismenzyme is selected from an oxalate decarboxylase, (e.g., OxdC (yvrK) and OxdD (yoaN) from Bacillus subtilis), an oxalyl-CoA decarboxylase (Oxc. e.g., from Lactobacillus acidophilus, or Bifidobacterium animalis), and a formyl-CoA transferase (e.g., Frc, e.g., from Lactobacillus acidophilus or Bifidobacterium animalis). In some embodiments, the at least one heterologous or endogenous gene encoding at least one oxalate catabolismenzyme is selected from an oxe gene. In some embodiments, the at least one heterologous or endogenous gene encoding at least one oxalate catabolismenzyme is selected from a fre gene and an oxe gene. In one embodiment, the at least one heterologous or endogenous gene encoding an oxalate transporter is an oxIT gene.

In some embodiments, the at least one heterologous or endogenous gene encoding at least one oxalate catabolismenzyme is located on a plasmid in the microbial cell. In some embodiments, the at least one heterologous or endogenous gene encoding at least one oxalate catabolismenzyme is located on a chromosome in the microbial cell. In some embodiments, the at least one heterologous or endogenous gene encoding an oxalate transporter is located on a plasmid in the microbial cell. In some embodiments, the at least one heterologous or endogenous gene encoding the oxalate transporter is located on a chromosome in the microbial cell. In some embodiments, the at least one heterologous or endogenous gene encoding a formate transporter is located on a plasmid in the microbial cell. In some embodiments, the at least one heterologous or endogenous gene encoding a formate transporter is located on a chromosome in the microbial cell. In some embodiments, the at least one heterologous or endogenous gene encoding an oxalate:formate antiporter is located on a plasmid in the microbial cell. In some embodiments, the at least one heterologous or endogenous gene encoding an oxalate:formate antiporter is located on a chromosome in the microbial cell.

In some embodiments, the engineered microbial cell is a probiotic microbial cell. In some embodiments, the engineered microbial cell is a member of a genus selected from the group consisting of Bacillus, Bifidobacterium, Clostridium, Escherichia, Lactobacillus and Lactococcus. In some embodiments, the recombinant microbial cell is of the species Bacillus subtilis. In some embodiments, the recombinant microbial cell is of the species Lactobacillus acidophilus. In some embodiments, the recombinant microbial cell is of the species Bifidobacterium animalis.

In another aspect, the invention provides a pharmaceutical composition comprising a recombinant microbial cell comprising at least one heterologous or endogenous gene encoding at least one oxalate catabolismenzyme operably linked to a first non-inducible promoter and a pharmaceutically acceptable carrier. In another aspect, the invention provides a pharmaceutical composition comprising a recombinant microbial cell comprising at least one heterologous or endogenous gene encoding at least one oxalate catabolismenzyme operably linked to a first non-inducible promoter, at least one heterologous or endogenous gene encoding an oxalate transporter operably linked to a second non-non-inducible promoter, which may be the same or a different promoter from the first non-inducible promoter, and a pharmaceutically acceptable carrier. In another aspect, the invention provides a pharmaceutical composition comprising a recombinant microbial cell comprising at least one heterologous or endogenous gene encoding at least one oxalate catabolism enzyme operably linked to a first non-inducible promoter, at least one heterologous or endogenous gene encoding a formate transporter operably linked to a second non-inducible promoter, which may be the same or different promoter from the first non-inducible promoter, and a pharmaceutically acceptable carrier. In another aspect, the invention provides a pharmaceutical composition comprising a recombinant microbial cell comprising at least one heterologous or endogenous gene encoding at least one oxalate catabolismenzyme operably linked to a first non-inducible promoter, at least one heterologous or endogenous gene encoding an oxalate:formate antiporter operably linked to a second non-inducible promoter, which may be the same or different promoter from the first non-inducible promoter, and a pharmaceutically acceptable carrier. In any of these embodiments, the first promoter and the second promoter may be separate copies of the same promoter. In another aspect, the invention provides a method for treating a disease or disorder in which oxalate is detrimental in a subject, the method comprising administering an engineered microbial cell or a pharmaceutical composition comprising an engineered microbial cell to the subject, wherein the engineered microbial cell comprises gene sequence encoding one or more oxalate catabolismenzyme(s). In another aspect, the invention provides a method for treating a disease or disorder in which oxalate is detrimental in a subject, the method comprising administering an engineered microbial cell or a pharmaceutical composition comprising an engineered microbial cell to the subject, wherein the engineered microbial cell comprises gene sequence encoding one or more oxalate transporter(s). In another aspect, the invention provides a method for treating a disease or disorder in which oxalate is detrimental in a subject, the method comprising administering an engineered microbial cell or a pharmaceutical composition comprising an engineered microbial cell to the subject, wherein the engineered microbial cell comprises gene sequence encoding one or more formate transporter(s). In another aspect, the invention provides a method for treating a disease or disorder in which oxalate is detrimental in a subject, the method comprising administering an engineered microbial cell or a pharmaceutical composition comprising an engineered microbial cell to the subject, wherein the engineered microbial cell comprises gene sequence encoding one or more oxalate:formate antiporter(s). In another aspect, the invention provides a method for treating a disease or disorder in which oxalate is detrimental in a subject, the method comprising administering an engineered microbial cell or a pharmaceutical composition comprising an engineered microbial cell to the subject, wherein the engineered microbial cell comprises gene sequence encoding one or more of the following: (i) oxalate catabolism enzyme(s); (ii) one or more oxalate transporter(s); (iii) one or more formate transporter(s); and (iv) one or more oxalate:formate antiporter(s).

In another aspect, the invention provides a method for treating a disease or disorder in which oxalate is detrimental in a subject, the method comprising administering an engineered microbial cell or a pharmaceutical composition comprising an engineered microbial cell to the subject, wherein the engineered microbial cell expresses at least one heterologous or endogenous gene encoding at least one oxalate catabolismenzyme in the subject, thereby treating the disease or disorder in which oxalate is detrimental in the subject. In some embodiments, the engineered microbial cell further expresses one or more of the following: (i) at least one heterologous or endogenous gene encoding an importer of oxalate; (ii) at least one heterologous or endogenous gene encoding a transporter of formate; and/or (iii) at least one heterologous or endogenous gene encoding an oxalate:formate antiporter. In one aspect, the invention provides a method for treating a disorder in which oxalate is detrimental in a subject, the method comprising administering an engineered microbial cell or a pharmaceutical composition of the invention to the subject, thereby treating the disorder in which oxalate is detrimental in the subject. In another aspect, the invention provides a method for decreasing a level of oxalate in plasma of a subject, the method comprising administering an engineered microbial cell or a pharmaceutical composition of the invention to the subject, thereby decreasing the level of oxalate in the plasma of the subject. In another aspect, the invention provides a method for decreasing a level of oxalate in urine of a subject, the method comprising administering an engineered microbial cell or a pharmaceutical composition of the invention to the subject, thereby decreasing the level of oxalate in the urine of the subject. In one embodiment, the level of oxalate is decreased in plasma of the subject after administering the engineered microbial cell or pharmaceutical composition to the subject. In another embodiment, the level of oxalate is reduced in urine of the subject after administering the engineered microbial cell or pharmaceutical composition to the subject. In one embodiment, the engineered microbial cell or pharmaceutical composition is administered orally. In another embodiment, the method further comprises isolating a plasma sample from the subject or a urine sample from the subject after administering the engineered microbial cell or pharmaceutical composition to the subject, and determining the level of oxalate in the plasma sample from the subject or the urine sample from the subject. In another embodiment, the method further comprises comparing the level of oxalate in the plasma sample from the subject or the urine sample from the subject to a control level of oxalate. In one embodiment, the control level of oxalate is the level of oxalate in the plasma of the subject or in the urine of the subject before administration of the engineered microbial cell or pharmaceutical composition.

In one embodiment, the disorder in which oxalate is detrimental is a hyperoxaluria. In one embodiment, the hyperoxaluria is primary hyperoxaluria type I. In another embodiment, the hyperoxaluria is primary hyperoxaluria type II. In another embodiment, the hyperoxaluria is primary hyperoxaluria type III. In one embodiment, the hyperoxaluria is enteric hyperoxaluria. In another embodiment, the hyperoxaluria is dietary hyperoxaluria. In another embodiment, the hyperoxaluria is idiopathic hyperoxaluria. In one embodiment, the disorder in which oxalate is detrimental is nephrocalcinosis. In one embodiment, the disorder in which oxalate is detrimental is urolithiasis. In one embodiment, the disorder in which oxalate is detrimental is nephrolithiasis.

In one embodiment, the subject consumes a meal within one hour of administering the pharmaceutical composition. In another embodiment, the subject consumes a meal concurrently with administering the pharmaceutical composition.

The present disclosure is based on the development of microbial cells, e.g., bacterial cells, that have been engineered to express one or more oxalate catabolismenzyme(s), or one or more oxalate catabolismenzyme(s) and one or more of the following: (i) one or more transporter(s) of oxalate; (ii) one or more transporter(s) of formate; (iii) one or more polypeptide(s) which mediate both the transport (import) of oxalate and the transport (export) of formate (e.g., oxalate:formate antiporter(s)); and (iv) any combination thereof, at levels exceeding the levels found in the non-engineered microbial cell.

According to embodiments of the present disclosure, the engineered microbial cells are fungal, microbial, or archaeal cells. According to embodiments of the present disclosure, the oxalate catabolismpolypeptide is located inside the cell (e.g., expressed in a microbial cell) and the oxalate transporter is located at the surface of the microbial cell, such that the oxalate transporter promotes uptake of oxalate into the cell. According to embodiments of the present disclosure, the oxalate catabolismpolypeptide is located extracellularly, e.g. at the surface of the microbial cell, such that the oxalate catabolismopolypeptide can degrade oxalate located outside the cell.

The engineered microbial cells of the present invention provide advantages to, for example, non-engineered cells. In contrast to the microbial cells of the present invention, which are engineered to comprise an oxalate catabolismpolypeptide inside the cell or outside the cell, e.g. on the surface of the cell, a non-engineered microbial cell is limited with respect to the levels of polypeptide that may be present in the cell or on the surface of the cell.

According to embodiments of the present disclosure, the engineered microbial cells are bacterial cells, e.g. the engineered microbial cell is a member of a genus selected from the group consisting of Bacillus, Bifidobacterium, Clostridium, Escherichia, Lactobacillus and Lactococcus. In some embodiments, the recombinant microbial cell is of the species Bacillus subtilis. In some embodiments, the recombinant microbial cell is derived from Bacillus subtilis strain PY79. In some embodiments, the recombinant microbial cell is derived from Bacillus subtilis subsp. inaquosorum strain DE111®. In some embodiments, the recombinant microbial cell is derived from Lactobacillus acidophilus strain La-14. In some embodiments, the recombinant microbial cell derived from Bifidobacterium animalis subsp. lactis

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a bar chart showing in vivo oxalate consumption by intact cells of various B. subtilis strains

### DETAILED DESCRIPTION OF THE INVENTION

In this Specification, which includes the figures, claims, and this detailed description, reference is made to particular and possible features of the embodiments of the invention, including method steps. These particular and possible features are intended to include all possible combinations of such features, without exclusivity. For instance, where a feature is disclosed in a specific embodiment or claim, that feature can also be used, to the extent possible, in combination with and/or in the context of other aspects and embodiments of the invention, and in the invention generally. Additionally, the disclosed architecture is sufficiently configurable, such that it may be utilized in ways other than what is shown.

The purpose of the Abstract of this Specification is to enable the U.S. Patent and Trademark Office and the public generally, and especially the scientists, engineers and practitioners of the art who are not familiar with patent or legal terms or phrasing, to determine quickly from a cursory inspection the nature and essence of the technical disclosure of the application. The Abstract is not intended to be limiting as to the scope of the invention in any way.

In the following description, numerous specific details are given in order to provide a thorough understanding of the present embodiments. It will be apparent, however, to one having ordinary skill in the art, that the specific detail need not be employed to practice the present embodiments. On other instances, well-known materials or methods have not been described in detail in order to avoid obscuring the present embodiments. When limitations are intended in this Specification, they are made with expressly limiting or exhaustive language.

Reference throughout this Specification to “one embodiment”, “an embodiment”, “one example” or “an example” means that a particular feature, structure, or characteristic described in connection with the embodiment or example is included in at least one embodiment of the present embodiments. Thus, appearances of the phrases “in one embodiment”, “according to an embodiment”, “in an embodiment”, “one example”, “for example”. “an example”, or the like, in various places throughout this Specification are not necessarily all referring to the same embodiment or example. Furthermore, the particular features, structures, or characteristics may be combined in any suitable combinations and/or sub-combinations in one or more embodiments or examples.

The terms “comprises”, “comprising”, “includes”, “including”, “has”, “having”, “could”, “could have” or their grammatical equivalents, are used in this Specification to mean that other features, components, materials, steps, etc. are optionally present as a non-exclusive inclusion. For instance, a device “comprising” (or “which comprises” or “is comprised of”) components A, B, and C can contain only components A, B, and C, or can contain not only components A, B, and C but also one or more other components. For example, a method comprising two or more defined steps can be carried out in any order or simultaneously, unless the context excludes that possibility; and the method can include one or more other steps which are carried out before any of the defined steps, between two of the defined steps, or after all the defined steps, unless the context excludes that possibility.

Further, unless expressly stated to the contrary, “or” refers to an inclusive or and not to an exclusive or. For example, An embodiment could have optional features A, B, or C, so the embodiment could be satisfied with A in one instance, with B in another instance, and with C in a third instance, and probably with AB, AC, BC, or ABC if the context of features does not exclude that possibility.

Examples or illustrations given are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these example or illustrations are utilized will encompass other embodiments, which may or may not be given in this Specification, and all such embodiments are intended to be included within the scope of that term or terms. Language designating such nonlimiting examples and illustrations includes, but is not limited to “for example”, “for instance”, “etc.”. “or otherwise”, and “in one embodiment.”

The phrase “at least” followed by a number is used to denote the start of a range beginning with that number, which may or may not be a range having an upper limit, depending on the variable defined. For instance, “at least 1” means 1 or more.

In this specification. “a” and “an” and similar phrases are to be interpreted as “at least one” and “one or more.” In this specification, the term “may” or “can be” or “could be” is to be interpreted as “may, for example.” In other words, the term “may” is indicative that the phrase following the term “may” is an example of one of a multitude of suitable possibilities that may, or may not, be employed to one or more of the various embodiments.

The phrase “a plurality of” followed by a feature, component, or structure is used to mean more than one, specifically including a great many, relative to the context of the component.

It is the applicant's intent that only claims that include the express language “means for” or “step for” be interpreted under 35 U.S.C. § 112. Claims that do not expressly include the phrase “means for” or “step for” are not to be interpreted under 35 U.S.C. § 112.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purpose required by law, but otherwise reserves all copyright rights whatsoever.

Many modifications and other embodiments of the inventions set forth herein will easily come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

As used herein, the terms “about”, “approximate”, and “substantially” when referring to a measurable value such as an amount, a temporal duration, and the like; would be understood by a person of ordinary skill in the art that the given feature is close enough to the exact feature or value that the invention can still be practiced; i.e., that the difference is not so significant as to render the present invention inoperable. From a quantifiable perspective, it might be helpful to think of these terms as encompassing variations of +20% or +10%, more preferably +5%, even more preferably +1%, and still more preferably +0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “such as”, “for example”. “e.g.” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

As used herein, a “microbial cell”, or “microbe” refers to single-celled organisms, whether organized as colonies, suspensions, individual cells, or other configurations and collections; alive or dead or in a state of metabolic stasis or suspension; including but not limited to organisms such as Bacteria, Archaea, Fungi, or Protists. Clearly, cells capable of enzymatic activity are needed to degrade oxalate, and thus practice the invention. For example, bacterial microbes may include e.g., Lactobacillus acidophilus, Bacillus subtilis, or Bifidobacterium animalis subsp. lactis, and fungal microbes may include e.g., Saccharomyces boulardii.

As used herein, an “additional therapeutic” refers to any therapeutic that is used in addition to another treatment. For example, when the method is one directed to treatment with the engineered microbial cells described herein, and the method comprises the use of an additional therapeutic, the additional therapeutic is in addition to the engineered microbial cells described herein. Generally, the additional therapeutic will be a different therapeutic. The additional therapeutic may be administered at the same time or at a different time and/or via the same mode of administration or via a different mode of administration, as that of the other therapeutic. In preferred embodiments, the additional therapeutic will be given at a time and in a way that will provide a benefit to the subject during the effective treatment window of the other therapeutic. When two compositions are administered with a specific time period, generally the time period is measured from the start of the first composition to the start of the second composition. As used herein, when two compositions are given within an hour, for example, the time before the start of the administration of the first composition is about an hour before the start of the administration of the second composition. In some embodiments, the additional therapeutic is another therapeutic for the treatment of hyperoxaluria, hyperoxalemia, nephrolithiasis, urolithiasis, or a condition associated with these. As used herein, a “hyperoxaluria, hyperoxalemia and/or urinary tract stone therapeutic” is any therapeutic that can be administered and from which a subject with hyperoxaluria, hyperoxalemia and/or urinary tract stones may derive a benefit because of its administration. In some embodiments, the hyperoxaluria, hyperoxalemia and/or urinary tract stone therapeutic is an oral therapeutic (i.e., a hyperoxaluria, hyperoxalemia and/or urinary tract stone therapeutic that can be taken or given orally, such as e.g. an alkalinizing agent).

As used herein, “dose” refers to a specific quantity of a pharmacologically active material for administration to a subject for a given time. Unless otherwise specified, the doses recited refer to an engineered microbial cell comprising an oxalate catabolismpolypeptide as described herein, an engineered microbial cell comprising an oxalate transporter as described herein, or an engineered microbial cell comprising an oxalate catabolismpolypeptide and an oxalate transporter as described herein. In some embodiments, a dose of engineered microbial cells refers to an effective amount of engineered microbial cells. For example, in some embodiments a dose or effective amount of engineered microbial cells comprises at least 10\hat{ }6 CFUs of engineered microbes per dose. In some embodiments, a dose or effective amount of engineered microbial cells refers to about 10\hat{ }6-10\hat{ }12 engineered microbial cells per dose. When referring to a dose for administration, in an embodiment of any one of the methods, compositions or kits provided herein, any one of the doses provided herein is the dose as it appears on a label/label dose.

As used herein, the term “endogenous” is meant to refer to a native form of compound (e.g., a small molecule) or process. For example, in some embodiments, the term “endogenous” refers to the native form of a nucleic acid or polypeptide in its natural location in the organism or in the genome of an organism.

As used herein, the term “an engineered cell” is meant to refer to a genetically-modified cell or progeny thereof.

As used herein, the term “microbial” cell refers to a cell, e.g., a bacterial, fungal or archacal cell, which may be a prokaryotic or eukaryotic cell. As used herein, a microbial cell includes a metabolically inactive spore or cell capable of germinating into or of being reconstituted into a metabolically active cell. As used herein, a microbial cell includes freeze-dried, spray-dried, or otherwise dried microbial cell.

As used herein, the term “probiotic” refers to live microbial cells that, when administered in adequate amounts, may or may not confer a health benefit on the host, and do not exert harmful effects.

As used herein, the term “exogenous,” when used in the context of nucleic acid, includes a transgene and engineered nucleic acids.

As used herein, the term “exogenous nucleic acid” refers to a nucleic acid (e.g., a gene) which is not native to a cell, but which is introduced into the cell or a progenitor of the cell. An exogenous nucleic acid may include a region or open reading frame (e.g., a gene) that is homologous to, or identical to, an endogenous nucleic acid native to the cell. In some embodiments, the exogenous nucleic acid comprises RNA. In some embodiments, the exogenous nucleic acid comprises DNA. In some embodiments, the exogenous nucleic acid is integrated into the genome of the cell. In some embodiments, the exogenous nucleic acid is processed by the cellular machinery to produce an exogenous polypeptide. In some embodiments, the exogenous nucleic acid is not retained by the cell or by a cell that is the progeny of the cell into which the exogenous nucleic acid was introduced.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide refers to a polypeptide that is introduced into or onto a cell, or is caused to be expressed by the cell by introducing an exogenous nucleic acid encoding the exogenous polypeptide into the cell or into a progenitor of the cell. In some embodiments, an exogenous polypeptide is a polypeptide encoded by an exogenous nucleic acid that was introduced into the cell, or a progenitor of the cell, which nucleic acid is optionally not retained by the cell.

As used herein, the term “express” or “expression” refers to the process to produce a polypeptide, including transcription and translation. Expression may be, e.g., increased by a number of approaches, including: increasing the number of genes encoding the polypeptide, increasing the transcription of the gene (such as by placing the gene under the control of a constitutive promoter), increasing the translation of the gene, knocking out of a competitive gene, or a combination of these and/or other approaches.

As used herein, the term “transcription regulatory sequence” refers to a first nucleotide sequence that regulates transcription of a second nucleotide sequence to which it is operatively linked.

A “promoter” is a transcription regulatory sequence at least sufficient to induce the transcription of a nucleotide sequence in DNA into an RNA transcript. A transcript transcribed from a promoter typically includes sequences from the promoter downstream of the transcription start site, as well as downstream sequences that, in the case of mRNA, encode an amino acid sequence. Promoters are the best-characterized transcriptional regulatory sequences because of their predictable location immediately upstream of transcription start sites. Promoters include sequences that modulate the recognition, binding and transcription initiation activity of the RNA polymerase. These sequences can be cis acting or can be responsive to trans acting factors. Promoters, depending upon the nature of the regulation, can be constitutive or regulated. They are often described as having two separate segments: core and extended promoter regions. Promotors may be unmodified and be identical to those found in nature, or be modified fr

The core promoter includes sequences that are sufficient for RNA polymerase recognition, binding and transcription initiation. The core promoter includes the transcriptional start site, an RNA polymerase binding site, and other general transcription binding sites and is where the pre-initiation complex forms and the general transcription machinery assembles. The pre-initiation complex is generally within 50 nucleotides (nt) of the transcription start site (TSS).

The core promoter also includes a sequence for a ribosome binding site, necessary for translation of an mRNA into a polypeptide.

The extended promoter region includes the so-called proximal promoter, which extends to about 250 nucleotides upstream of the transcriptional start site (i.e. , −250 nt). It includes primary regulatory elements such as specific transcription factor binding sites. It has been found that many genes have transcription regulatory elements located further upstream. In particular, a fragment that includes most of the transcription regulatory elements of a gene can extend up to 700 nt or more up-stream of the transcription start site. In certain genes, transcription regulatory sequences have been found thousands of nucleotides upstream of the transcriptional start site.

Promoters can be inducible or non-inducible. Inducible promoters cause transcription of downstream sequences if certain environmental conditions are being met. For example, an oxygen-sensitive inducible promoter can cause transcription of downstream sequences if oxygen levels fall below a certain concentration. Non-inducible promoters cause transcription of downstream sequences regardless of environmental conditions. For example, a non-inducible promoter may cause transcription of downstream sequences regardless of oxygen levels. Non-inducible promoters are also known as constitutive promoters, and will cause transcription of downstream sequences in all circumstances.

The rate at which relevant environmental conditions change and cross the particular threshold at which an inducible promoter induced by those conditions starts to cause transcription of downstream gene sequence(s), as well as the responsiveness of that specific inducible promoter to the change in relevant environmental condition will determine the rate at which downstream gene sequence(s) encoding one or more enzyme(s) or other protein(s) of interest will be transcribed and accumulate. Consequently, the rate at which the one or more enzyme(s) or other protein(s) of interest levels controlled by an inducible promoter rise, as well as the levels to which they rise, depends on the rate at which environmental conditions that control the inducible promoter change, as well as the final level of environmental conditions that control the inducible promoter. For example, a promoter that is directly or indirectly induced by low-oxygen or anaerobic conditions, wherein expression of the downstream gene sequence(s) encoding one or more enzyme(s) or other protein(s) of interest is activated under low-oxygen or anaerobic environments, such as the environment of the mammalian gut, may respond slowly upon reaching activating conditions (low-oxygen or anaerobic conditions), and may cause transcription of downstream genes sequence(s) to initiate slowly and rise to a non-maximal level if oxygen tension remains above the level that is sufficiently low to cause maximal transcription levels.

In contrast, non-inducible, constitutive promoters provide maximal levels of transcription of downstream gene sequence(s) encoding one or more enzyme(s) or other protein(s) of interest, and consequently levels of enzyme(s) or other protein(s) of interest under all environmental conditions. For example, a non-inducible, constitutive promoter that causes transcription of downstream gene sequence(s) encoding one or more enzyme(s) or protein(s) of interest is active under all conditions, independent of oxygen tension or anaerobic status of the environment, including the environment of the mammalian gut, and will cause transcription of downstream gene sequence(s) encoding one or more enzyme(s) or other protein(s) of interest at maximal levels. Consequently, levels of the one or more enzyme(s) or other protein(s) of interest are always maximal.

To obtain optimal benefit from a desired metabolic function provided by a microbial cell or cells, e.g. oxalate catabolismgenes sequence(s) encoding the metabolic function, e.g. gene sequence(s) encoding oxalate catabolismenzymes, must be transcribed at the highest obtainable level under all conditions, and not be subject to limitations depending on the rate at which environmental conditions change and cross a certain threshold, as well as the responsiveness of the specific inducible promoter, or the final level of environmental conditions that control an inducible promoter.

Thus, use of strong, non-inducible, constitutive promoters to drive transcription of downstream genes sequence(s) encoding one or more enzyme(s) or other protein(s) of interest that form (part of) a desired metabolic function is a superior choice for probiotics providing a desired metabolic function, e.g. oxalate catabolismas compared to the use of various inducible promoters.

As used herein, a nucleotide sequence is “operatively linked” or “operably linked” with a transcription regulatory sequence when the transcription regulatory sequence functions in a cell to regulate transcription of the nucleotide sequence. This includes promoting transcription of the nucleotide sequence through an interaction between a polymerase and a promoter.

As used herein, a first nucleotide sequence is “heterologous or endogenous” to a second nucleotide sequence if the first nucleotide sequence is not operatively linked with the second nucleotide sequence in nature. By extension, a polypeptide is “heterologous or endogenous” to an expression control sequence if it is encoded by nucleotide sequence heterologous or endogenous the promoter.

As used herein, the terms “first”, “second” and “third”, etc. with respect to exogenous polypeptides are used for convenience of distinguishing when there is more than one type of exogenous polypeptide. Use of these terms is not intended to confer a specific order or orientation of the exogenous polypeptides unless explicitly so stated.

As used herein, the term “fragment” refers to sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full-length sequence. Fragments may be derived from any contiguous portion of a nucleic acid or amino acid sequence of choice.

As used herein, the term “gene” is used broadly to refer to any segment of nucleic acid associated with expression of a given RNA or protein. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

As used herein the term “nucleic acid molecule” refers to a single or double- stranded polymer of deoxyribonucleotide or ribonucleotide bases. It includes chromosomal DNA and self-replicating plasmids, vectors, mRNA, tRNA, siRNA, etc. which may be engineered and from which exogenous polypeptides may be expressed when the nucleic acid is introduced into a cell.

The following terms are used herein to describe the sequence relationships between two or more nucleic acids or polynucleotides: (a) “reference sequence”, (b) “comparison window”. (c) “sequence identity”, (d) “percentage of sequence identity”, and (e) “substantial identity.”

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches. Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif .; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, Nucleic Acids Research 16:10881-90 (1988); Huang, et al., CABIOS, 8:155-65 (1992), and Pearson, et al., Methods in Molecular Biology, 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19. Ausubel, et al, Eds., Greene Publishing and Wiley-Interscience, New York (2003). Unless otherwise stated, sequence identity/similarity values provided herein refer to the value obtained using the BLAST 2.0 suite of programs using default parameters. Altschul et al, Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always O). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative scoring residue alignments; or the end of either sequence is reached. The BLAST algorithm parameters W. T. and X determine the sensitivity and speed of the alignment. The BLASTN program (for nucleotide sequences) uses as defaults a word length (W) of 11, an expectation (E) of 10, a cutoff of 100, M=5, N =- 4, and a comparison of both strands. For amino acid sequences, the BLASTP program uses as defaults a word length (W) of 3, an expectation (E) of 10, and the BLOSUM62 scoring matrix (see Henikoff and Henikoff Proc. Natl. Acad. Sci. USA 89.10915 (1989). In addition to calculating percent sequence identity, the BLAST algorithm also performs a statistical analysis of the similarity between two sequences (see, e.g., Karlin and Altschul, Proc. Natl. Acad. Sci. USA 90:5873-5887 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wootton and Federhen, Comput. Chem., 17:149-163 (1993)) and XNU (Claverie and States, Comput. Chem., 17:191-201 (1993)) low-complexity filters may be employed alone or in combination

The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Myers and Miller, CABIOS, 4:11-17 (1988) e.g., as implemented in the program PC/GENE (Intelligenetics, Mountain View, Calif., USA)

The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, at least 80% sequence identity, at least 90% sequence identity or at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid. Mutations may also be made to the nucleotide sequences of the present proteins by reference to the genetic code. including taking into account codon degeneracy.

As used herein, the term “probiotic composition” refers to a composition comprising probiotic microorganisms and a physiologically acceptable carrier. Typically, a probiotic composition confers a health or wellness benefit on the host subject to whom it is administered.

As used herein, the term “physiologically acceptable” refers to a carrier that is compatible with the other ingredients of a composition and can be safely administered to a subject. Probiotic compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al. . “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The probiotic composition may be a liquid formulation or a solid formulation. When the probiotic composition is a solid formulation it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip or a film. When the probiotic composition is a liquid formulation it may be formulated as an oral solution, a suspension, an emulsion or syrup. Said composition may further comprise a carrier material independently selected from, but not limited to, the group consisting of lactic acid fermented foods, fermented dairy products, resistant starch, dietary fibers, carbohydrates, proteins, and glycosylated proteins.

As used herein, the probiotic composition could be formulated as a food composition, a dietary supplement, a functional food, a medical food or a nutritional product as long as the required effect is achieved, e.g. treatment or prevention of an alcohol hangover. Said food composition may be chosen from the group consisting of beverages, yogurts, juices, ice creams, breads, biscuits, crackers, cereals, health bars, spreads, gummies and nutritional products. The food composition may further comprise a carrier material, wherein said carrier material is chosen from the group consisting of lactic acid fermented foods, fermented dairy products, resistant starch, dietary fibers, carbohydrates, proteins and glycosylated proteins. As used herein, the terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural processes and by entirely synthetic methods, as well. According to some embodiments, the peptide is of any length or size.

As used herein, polypeptides referred to herein as “engineered” refers to polypeptides which have been produced by engineered DNA methodology, including those that are generated by procedures which rely upon a method of artificial recombination, such as the polymerase chain reaction (PCR) and/or cloning into a vector using restriction enzymes.

“Engineered” polypeptides are also polypeptides having altered expression, such as a naturally occurring polypeptide with engineeredly modified expression in a cell, such as a host cell.

As used herein, the terms “subject”, “individual”, “host”, “recipient”, “person”, and “patient” are used interchangeably herein and refer to any mammalian subject for whom diagnosis, treatment. or therapy is desired, particularly humans. The methods described herein are applicable to both human therapy and veterinary applications. In some embodiments, the subject is a mammal, and in particular embodiments the subject is a human.

As used herein, the phrase “subject in need” refers to a subject that (i) will be administered an engineered microbial cell (or pharmaceutical composition comprising an engineered microbial cell) according to the described invention, (ii) is receiving an engineered microbial cell (or pharmaceutical composition comprising an engineered microbial cell) according to the described invention; or (iii) has received an engineered microbial cell (or pharmaceutical composition comprising an engineered microbial cell) according to the described invention; or (iv) is in need of and/or would benefit from administration of an engineered microbial cell (or pharmaceutical composition comprising an engineered microbial cell) according to the described invention, unless the context and usage of the phrase indicates otherwise

As used herein, the term “suppress”, “decrease”, “interfere”, “inhibit” and/or “reduce” (and like terms) generally refers to the act of reducing, either directly or indirectly, a concentration, level, function, activity, or behavior relative to the natural, expected, or average, or relative to a control condition.

As used herein, the terms “therapeutic amount”, “therapeutically effective amount”, an “amount effective”, or “pharmaceutically effective amount” of an active agent (e.g. an engineered microbial cell as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 12th Edition, McGraw-Hill (New York) (2001) are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

As used herein, the terms “treat”, “treating”, and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, a “disorder in which oxalate is detrimental” is a disease or disorder involving the abnormal, e.g., increased, levels of oxalate and/or oxalic acid or molecules directly upstream, such as glyoxylate. In one embodiment, the disorder in which oxalate is detrimental is a disorder or disease in which hyperoxaluria is observed in the subject. In one embodiment the disorder in which oxalate is detrimental refers to any condition(s), disorder(s), disease(s), predisposition(s), and/or genetic mutations(s) that result in daily urinary oxalate excretion over 25 mg per 24 hours. In one embodiment the disorder in which oxalate is detrimental is a disorder or disease selected from the group consisting of: PHI, PHII, PHII, secondary hyperoxaluria, enteric hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, hyperoxalemia, calcium oxalate nephrocalcinosis, calcium oxalate nephrolithiasis, and calcium oxalate urolithiasis. Such disorders may optionally be acute or chronic. Elevated levels refer to levels that are higher than levels that are considered normal by the American Medical Association,

As used herein, “oxalic acid”, also known as oxalate (the two terms are used interchangeably herein), refers to a metabolic end product of mammalian metabolism. Humans produce substantial quantities of oxalate. Oxalate concentrations can be measured in samples from a subject, e.g., blood or urine samples, using known methods.

As used herein, an “oxalate catabolismenzyme”, “oxalate catabolismpolypeptide”, “oxalate catabolismpolypeptide” or “oxalate catabolismenzyme” refers to any polypeptide (enzyme) that is involved in catabolizing or degrading oxalate. Examples of oxalate catabolismenzymes include oxalate decarboxylase (EC 4.1.1.2), oxalate oxidase (EC 1.2.3.4), formyl coenzyme A transferase (also known as formyl-CoA transferase, EC 2.8.3.16), oxalyl coenzyme A decarboxylase (also known as Oxalyl-CoA decarboxylase, EC 4.1.1.8), oxalate coenzyme A ligase (also known as oxalate CoA ligase, EC 6.2.1.8), oxalate oxidoreductase, EC 1.2.7.10), oxalate coenzyme A-transferase (also known as oxalate CoA-transferase, EC 2.8.3.2), acetyl-coenzyme A:oxalate coenzyme A-transferase (also known as acetyl-CoA oxalate CoA-transferase, EC 2.8.3.19).

Other examples of oxalate catabolismpolypeptides are described herein and are not intended to be limiting. In an embodiment, an oxalate catabolismpolypeptide has oxalate as its substrate, or one of its substrates. In an embodiment, an oxalate catabolismpolypeptide catalyzes the decarboxylation of oxalate.

As used herein, “oxalate degrading activity” refers to the removal of free oxalate from a solution. Oxalate degrading activity is measured in units, where one unit of activity is defined as the removal of 1 umol of oxalate per minute. In an embodiment, an oxalate catabolismpolypeptide alone has oxalate degrading activity. In an embodiment, two or more oxalate catabolismpolypeptides contribute to oxalate degrading activity.

Oxalate may be catabolized (i.e. consumed by enzymatically catalyzed reactions) intracellularly or extracellularly (e.g. by oxalate catabolismenzymes located on the cell wall or by oxalate catabolismenzymes that are secreted and not bound to the cell).

Intracellular oxalate catabolismenzymes include for example, but are not limited to oxalate oxidase (EC 1.2.3.4), formyl coenzyme A transferase (also known as formyl-CoA transferase, EC 2.8.3.16), oxalyl coenzyme A decarboxylase (also known as Oxalyl-CoA decarboxylase, EC 4.1.1.8), oxalate coenzyme A ligase (also known as oxalate CoA ligase, EC 6.2.1.8), oxalate oxidoreductase (EC 1.2.7.10), oxalate coenzyme A-transferase (also known as oxalate CoA-transferase, EC 2.8.3.2), and acetyl-coenzyme A:oxalate coenzyme A-transferase (also known as acetyl-CoA oxalate CoA-transferase, EC 2.8.3.19). Besides consuming oxalate, reactions catalyzed by intracellular oxalate catabolismenzymes require (regeneration or production of) other substrates, e.g. Coenzyme A or conjugates thereof (e.g. formyl-CoA), oxidized ferredoxin, molecular oxygen (O₂), or other substrates, which can limit overall oxalate catabolismrates if reaction substrates other than oxalate are present at a limiting concentration within the cell.

Reactions catalyzed by intracellular oxalate catabolismenzymes are coupled with other reactions. For example, Oxalyl-CoA decarboxylase (EC 4.1.1.8) is coupled with formyl coenzyme A (CoA) transferase (EC 2.8.3.16). The reaction catalyzed by formyl coenzyme A (CoA) transferase, and the concurrent catabolismconsumption) of oxalate, depends on the regeneration of the substrate formyl coenzyme A by the coupled enzyme Oxalyl-CoA decarboxylase. The relative reaction rates of such coupled reactions can limit the overall rate of oxalate catabolismcatalyzed by intracellular oxalate catabolismenzymes by limiting the availability of intracellular substrates other than oxalate participating in the reactions catalyzed by intracellular oxalate catabolismenzymes. Reactions catalyzed by intracellular oxalate catabolismenzymes require transporters (importers) of oxalate, which can potentially limit the overall rate of oxalate catabolismreactions catalyzed by intracellular oxalate catabolismenzymes by limiting the availability of intracellular oxalate. Reactions catalyzed by intracellular oxalate catabolismenzymes require transporters (exporters) of formate, which can potentially limit the overall rate of oxalate catabolismreactions catalyzed by intracellular oxalate catabolismenzymes by allowing for excess (inhibitory levels of) formate to accumulate intracellularly.

Extracellular oxalate catabolismenzymes include for example, but are not limited to oxalate decarboxylase (EC 4.1.1.2). Besides consuming oxalate, reactions catalyzed by extracellular oxalate catabolismenzymes (i.e. oxalate decarboxylase, EC 4.1.1.2) only consume H⁺ (protons). Unlike reactions catalyzed by intracellular oxalate catabolismenzymes, reactions catalyzed by extracellular oxalate catabolismenzymes do not require (regeneration or production of) additional substrates, e.g. Coenzyme A or conjugates thereof (e.g. formyl-CoA), oxidized ferredoxin, molecular oxygen (O₂), or other substrates. Thus, oxalate catabolismrates obtained with microbial cells employing extracellular oxalate catabolismenzymes (i.e. oxalate decarboxylase, EC 4.1.1.2) have far fewer limitations than oxalate catabolismrates obtained with microbial cells employing intracellular oxalate catabolism enzymes

Reactions catalyzed by extracellular oxalate catabolismenzymes (i.e. oxalate decarboxylase, EC 4.1.1.2) are not coupled with other reactions. Reactions catalyzed by extracellular oxalate catabolismenzymes (i.e. oxalate decarboxylase, EC 4.1.1.2) do not require transporters (importers) of oxalate, or transporters (exporters) of formate for their function.

Thus, extracellular oxalate catabolismenzymes (i.e. oxalate decarboxylase, EC 4.1.1.2) have fewer potential restrictions that could reduce the rate of oxalate catabolismas compared to intracellular oxalate catabolismenzymes. Thus, for the purposes of engineering microbes that provide the highest levels of oxalate catabolismthe use of (engineering of expression levels of) extracellular oxalate catabolismenzymes (i.e. oxalate decarboxylase, EC 4.1.1.2) is preferred over the use of intracellular oxalate catabolismenzymes.As used herein, a “transporter of oxalate” refers to any polypeptide that facilitates the movement of oxalate across cell membranes or cell walls. Such movement may be driven exclusively by a concentration gradient of oxalate (passive diffusion). Alternatively, the movement may be driven by or facilitated by concertation gradients of other solutes (active transport). For example, a formate or proton concentration gradient may enhance transport rates of oxalate.

As used herein, a “transporter of formate” refers to any polypeptide that facilitates the movement of formate across cell membranes or cell walls. Such movement may be driven exclusively by a concentration gradient of formate (passive diffusion). Alternatively, the movement may be driven by or facilitated by concertation gradients of other solutes (active transport). For example, an oxalate or proton concentration gradient may enhance transport rates of formate.

As used herein, a “polypeptide which mediate both the transport (import) of oxalate and the transport (export) of formate (e.g., oxalate:formate antiporter(s));” refers to any polypeptide that facilitates the movement of both oxalate and formate across cell membranes or cell walls. Such movement may be in the form of antiport, where the import of one molecule of oxalate occurs in exchange for the export of one molecule of formate, and where both are facilitated by the same polypeptide.

As used herein, the term “variant” refers to a polypeptide which differs from the original protein from which it was derived (e.g., a wild-type protein) by one or more amino acid substitutions, deletions, insertions (i.e., additions), or other modifications. In some embodiments, these modifications do not significantly change the biological activity of the original protein. In many cases, a variant retains at least 10%, 20%, 30%, 40%, 50%, 60%, 70%, 80%, 90%, 95%, or 100% of the biological activity of original protein. The biological activity of a variant can also be higher than that of the original protein. A variant can be naturally-occurring, such as by allelic variation or polymorphism, or be deliberately engineered. For example, a variant may comprise a substitution at one or more amino acid residue positions to replace a naturally-occurring amino acid residue for a structurally similar amino acid residue. Structurally similar amino acids include: (I, L and V); (F and Y); (K and R); (Q and N); (D and E); and (G and A). In some embodiments, variants include (i) polymorphic variants and natural or artificial mutants, (ii) modified polypeptides in which one or more residues is modified, and (iii) mutants comprising one or more modified residues. Variants may differ from the reference sequence (e.g., by truncation, deletion, substitution, or addition) by no more than 1, 2, 3, 4, 5, 8, 10, 20, or 50 residues, and retains (or encodes a polypeptide that retains) a function of the wild-type protein from which they were derived.

The amino acid sequence of a variant is substantially identical to that of the original protein. In many embodiments, a variant shares at least 50%, 60%, 70%, 75%, 80%, 85%, 90%, 91%, 92%, 93%, 94%, 95%, 96%, 97%, 98%, or 99%, or more global sequence identity or similarity with the original protein. Sequence identity or similarity can be determined using various methods known in the art, such as Basic Local Alignment Tool (BLAST), dot matrix analysis, or the dynamic programming method. In one example, the sequence identity or similarity is determined by using the Genetics Computer Group (GCG) programs GAP (Needleman-Wunsch algorithm) The amino acid sequences of a variant and the original protein can be substantially identical in one or more regions, but divergent in other regions. A variant may include a fragment (e.g., a biologically active fragment of a polypeptide). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide.

### Engineered Microbial Cells

The present disclosure features engineered microbial cells that are engineered to include at least one exogenous polypeptide comprising an oxalate catabolismpolypeptide, an oxalate transporter, or both. In some embodiments the microbial cell is a bacterial cell.

The present disclosure provides microbial cells that are engineered to degrade oxalate by expression of an oxalate catabolismpolypeptide, an oxalate transporter, or both an oxalate catabolism polypeptide and an oxalate transporter. In some embodiments the microbial cell is a bacterial cell.

The engineered cells may be advantageously used to reduce the oxalate concentration in the milieu surrounding the cell (e.g., in vitro or in vivo). For example, the engineered cells provided herein may be administered to a subject (e.g., a human subject) to reduce the concentration of oxalate in the subject (e.g., in the intestinal lumen, in the digestive tract, blood, plasma, serum, or urine of the subject, or elsewhere in the subject).

In some embodiments, the disclosure provides an engineered microbial cell comprising at least one (e.g., one, two, three, four, or more) exogenous polypeptides, wherein each exogenous polypeptide may comprise either at least one oxalate catabolismpolypeptide, at least one oxalate transporter, or both a oxalate catabolismpolypeptide and a oxalate transporter.

Any condition, disease or disorder in which a reduction of oxalate levels is desired may be treated by administering the engineered cells provided herein.

In some embodiments of any of the aspects herein, the engineered microbial cell is a bacterial cell.

### Oxalate Catabolisme Enzymes

In one aspect, the present disclosure provides a microbial cell engineered to degrade oxalate, comprising an exogenous polypeptide comprising at least one oxalate catabolismpolypeptide, or a variant thereof. In some embodiments, the microbial cell comprises more than one (e.g., two, three, four, five, or more) exogenous polypeptides, each comprising at least one oxalate catabolism polypeptide, or a variant thereof. In some embodiments, the engineered cells described herein comprise more than one type of exogenous polypeptide, wherein each exogenous polypeptide comprises an oxalate catabolismpolypeptide, and wherein the oxalate catabolismpolypeptides are not the same (e.g., the oxalate catabolismpolypeptides may comprise different types of oxalate catabolismo polypeptides, or variants of the same type of oxalate catabolismpolypeptide). For example, in some embodiments, the engineered cell may comprise a first exogenous polypeptide comprising formyl-coenzyme A (CoA) transferase (Frc), or a variant thereof, and a second exogenous polypeptide comprising a second oxalate catabolismpolypeptide that is not a formyl-coenzyme A (CoA) transferase, such as for example a oxalyl-CoA decarboxylase (Oxc). In addition, an exogenous polypeptide may comprise more than one (e.g., one, two, three, four, five, or more) oxalate catabolism polypeptides (e.g., two different formyl-coenzyme A (CoA) transferases).

Many oxalate catabolismpolypeptides are known in the art and may be used as described herein. Any one or more of the enzymes involved in oxalate catabolism (i.e., oxalate catabolism polypeptides) can be included in the microbial cells described herein.

In some embodiments, the at least one oxalate catabolismpolypeptide, or variant thereof, can be derived from any source or species, e.g., fungal, plant or microbial sources, or can be engineered. In some embodiments, the oxalate catabolismpolypeptide can be a chimeric oxalate catabolism polypeptide, e.g., derived from two different species.

The exogenous polypeptides included in the engineered cells provided herein may comprise an exogenous polypeptide comprising any oxalate catabolismpolypeptide. In some embodiments, the oxalate catabolismpolypeptide comprises a oxalate decarboxylase, or a variant thereof.

Oxalate decarboxylases, and variants thereof, are described in detail below.

In some embodiments, the oxalate catabolismpolypeptide comprises or consists of a variant of the wild-type oxalate catabolismpolypeptide having at least 60%, sequence identity to the amino acid sequence of a corresponding wild-type oxalate catabolismpolypeptide.

### Oxalate Decarboxylases

Oxalate therapies (e.g., low-oxalate or low-fat diet, pyridoxine, adequate calcium, and increased fluids), are only partially effective and they may have undesirable adverse side effects, such as the gastrointestinal effects of orthophosphate, magnesium, or cholestyramine supplementation and the risks of dialysis and surgery. Accordingly, methods that safely remove oxalate from the body are needed. Moreover, methods that degrade oxalate to reduce oxalate levels in a biological sample are advantageous over a therapy, for example, that solely blocks absorption or increased clearance of oxalate.

The disclosure provides, in one aspect, a microbial cell engineered to degrade oxalate, comprising a first exogenous polypeptide comprising an oxalate decarboxylase, or a variant thereof. In some embodiments, the microbial cell comprises more than one (e.g., two, three, four, or five) exogenous polypeptide comprising an oxalate decarboxylase.

Oxalate decarboxylase (also referred to as Oxd or ODC) is a manganese-dependent, multimeric enzyme of the cupin protein superfamily. Oxalate decarboxylase (EC 4.1.1.2) catalyzes the chemical reaction oxalate+H⁺→formate+CO₂. This enzyme belongs to the family of lyases, specifically the carboxy-lyases, which cleave carbon-carbon bonds. The systematic name of this enzyme class is oxalate carboxy-lyase (formate-forming). Oxalate decarboxylase also referred to as oxalate carboxy-lyase.

Mammals do not have oxalate decarboxylase, but it is found in fungi, yeast, and bacteria. As a consequence, in susceptible individuals, excessive concentrations of oxalate in the blood (hyperoxalemia) and in the urine (hyperoxaluria) can lead to various disorders as described herein.

Unlike other oxalate catabolismenzymes, which are located intracellularly, oxalate decarboxylase is located extracellularly. Oxalate decarboxylase enzymes function outside the cell, e.g. on the surface of a microbial cell. The reaction catalyzed by oxalate decarboxylase, is not directly coupled with other reactions. Because the reaction catalyzed by oxalate decarboxylase is extracellular, oxalate catabolismcatalyzed by oxalate decarboxylase does not require functional expression of transporters (importers) of oxalate, or transporters (exporters) of formate.

In certain embodiments, the oxalate catabolismenzyme may be located intracellularly. Surprisingly, cells overexpressing B. subtilis oxalate decarboxylase, but not non-engineered parental cells, were able to convert oxalate in a solution to highly soluble formate.

This was unexpected as B. subtilis oxalate decarboxylase is known to be an intracellular enzyme, as these cells were not engineered to overexpress oxalate and formate transporters or oxalate:formate antiporters, and as the assay detects the formation of formate, which is the product of the oxalate decarboxylase reaction.

An engineered microbial cell of the disclosure may comprise an exogenous polypeptide comprising an oxalate decarboxylase, or variant thereof, wherein the oxalate decarboxylase is derived from any source(s) known in the art, including fungal or bacterial, or other microbial sources, as well as by engineered technologies.

In some embodiments, the oxalate decarboxylase, or oxalate decarboxylase variant, is obtained from a bacterial source. In some embodiments, the oxalate decarboxylase is derived from Bacillus subtilis. In some embodiments, the oxalate decarboxylase is Bacillus subtilis OxdC (SEQ ID NO: [symbol] or SEQ ID NO: [symbol] ). In some embodiments, the oxalate decarboxylase is Bacillus subtilis OxdD (SEQ ID NO: [symbol] or SEQ ID NO [symbol] ) In particular embodiments the oxalate decarboxylase comprises at least 95% identity with the full length of any one of SEQ ID NOs: 1-4.). In further embodiments the oxalate decarboxylase comprises at least 90% identity with the full length of any one of SEQ ID NOs: 1-4.

In some embodiments, the oxalate decarboxylase, or oxalate decarboxylase variant, is derived from a fungus, for example, Collybia velutipes, Coriolus hersutu, or Sclerotinia sclerotiorum

In some embodiments, the oxalate decarboxylase is a chimeric oxalate decarboxylase, in which portions of the oxalate decarboxylase are derived from different sources. For example, a portion of the chimeric oxalate decarboxylase may be obtained (e.g., derived) from one organism and one or more other portions of the chimeric oxalate decarboxylase may be obtained (e.g., derived) from another organism.

In some preferred embodiments of the disclosure, the oxalate decarboxylase (or variant thereof) comprises or consists of an oxalate decarboxylase selected from those set forth in Table 1. below, including one or more oxalate decarboxylases derived from Bacillus subtilis. In some embodiments. the oxalate decarboxylase comprises an amino acid sequence of SEQ ID NO: [symbol] or SEQ ID NO: [symbol] ). In some embodiments, the oxalate decarboxylase decarboxylase comprises an amino acid sequence of SEQ ID NO: [symbol] or SEQ ID NO [symbol] . In some embodiments, the oxalate decarboxylase comprises the Bacillus subtilis oxalate decarboxylase comprising the amino acid sequence set forth in SEQ ID NO: [symbol] . In some embodiments, the oxalate decarboxylase comprises the Bacillus subtilis oxalate decarboxylase comprising the amino acid sequence set forth in SEQ ID NO: [symbol] . In some embodiments, the oxalate decarboxylase comprises the Bacillus subtilis oxalate decarboxylase comprising the amino acid sequence set forth in SEQ ID NO: [symbol] . In some embodiments, the oxalate decarboxylase comprises the Bacillus subtilis oxalate decarboxylase comprising the amino acid sequence set forth in SEQ ID NO: [symbol] .

In some embodiments, the oxalate decarboxylase comprises a variant of a wild-type oxalate decarboxylase having at least 60% sequence identity to the amino acid sequence of any one of SEQ ID NO: [symbol] or SEQ ID NO: [symbol] or SEQ ID NO: [symbol] or SEQ ID NO: [symbol] . In some embodiments, the oxalate decarboxylase variant possesses a function of the oxalate decarboxylase from which it was derived (e.g., the ability to catalyze the decarboxylation of oxalate to formate.

In a particular embodiment, the oxalate decarboxylase consists of the amino acid sequence of any one of SEQ ID NO: [symbol] or SEQ ID NO: [symbol] or SEQ ID NO: [symbol] or SEQ ID NO: [symbol] .

In general. a variant oxalate decarboxylase, from any origin, may be produced, for example. to enhance production of the protein in an engineered cell. to improve turnover/half-life of the protein or mRNA encoding the protein, and/or to modulate (enhance or reduce) the enzymatic activity of the oxalate decarboxylase. The oxalate decarboxylase. whatever the source, may also be in a form that is truncated, either at the amino terminal, or at the carboxyl terminal, or at both terminals.

In some embodiments, the invention provides an engineered microbial cell (e.g. an engineered bacterial cell) comprising a nucleic acid sequence encoding an oxalate catabolismpolypeptide as described herein. In some embodiments. the invention provides an engineered microbial cell prepared by using a nucleic acid sequence encoding an oxalate catabolismpolypeptide (e.g. an oxalate decarboxylase) as described herein. In some embodiments, the nucleic acid sequence encodes an oxalate decarboxylase as described herein.

In some embodiments, the exogenous polypeptide is a fusion polypeptide comprising an oxalate decarboxylase, or a variant thereof, linked to a heterologous or endogenous protein sequence (e.g., via a linker).

### Oxalate Transporters

In one aspect, the disclosure provides an engineered microbial cell comprising a first exogenous polypeptide comprising an oxalate transporter, or a variant thereof. In some embodiments, the disclosure provides an engineered microbial cell comprising at least one (e.g., one, two, three, four, or more) exogenous polypeptide comprising an oxalate transporter. In some embodiments, the disclosure provides an engineered microbial cell comprising more than one exogenous polypeptide, each comprising an oxalate transporter.

In another aspect, the disclosure provides a microbial cell engineered to degrade oxalate, wherein the cell comprises a first exogenous polypeptide comprising an oxalate catabolism polypeptide, e.g., oxalate decarboxylase, or a variant thereof, and further comprises a second exogenous polypeptide comprising an oxalate transporter, or a variant thereof.

In yet another aspect, the disclosure provides an engineered cell comprising at least one (e.g., one two, three, four, or more) exogenous polypeptide, wherein the exogenous polypeptide comprises both an oxalate catabolismpolypeptide (or a variant thereof) and an oxalate transporter (or a variant thereof). Without wishing to be bound by any particular theory, engineered cells comprising an exogenous polypeptide that comprises both an oxalate catabolismpolypeptide and an oxalate transporter may improve turnover of oxalate (e.g., the biodegradation of oxalate) by facilitating the transfer of oxalate from the oxalate transporter to the oxalate catabolismpolypeptide, thereby microcompartmentalizing the channeling and biodegradation of oxalate.

In humans, oxalate transporters regulate oxalate uptake in the gastrointestinal tract and excretion in the kidney and thereby regulate oxalate levels in bodily fluids such as serum and urine.

In microbes, oxalate transporters contribute to the uptake of oxalate from the environment (Iyalomhe, Khantwal, & Kang, 2015). Typically, microbial oxalate transporters enable the use of oxalate as a carbon and/or as an energy source, enabling growth and division of the microbe (Anantharam, Allison, & Maloney, 1989)

Microbial oxalate transporters include archaeal, fungal, and bacterial oxalate transporters. It is expected any microbial organisms whose genome encodes gene products predicted to be involved in intracellular oxalate degradation (e.g., Oxalyl-CoA decarboxylase) will also have oxalate transporters encoded in their genome. In some prokaryotes, these would be multipass transmembrane proteins encoded in the same operon that encodes the Oxalyl-CoA decarboxylase.

Well characterized and potential microbial oxalate transporters that could be used to engineer microbial cells of the present invention in order to improve oxalate transport and facilitate oxalate degradation include, but are not limited to: Lactobacillus acidophilus strain La-14 WP_015613377. Bifidobacterium animalis subsp. Lactis BAH23805, and Oxalobacter formigenes Oxc.

In some embodiments, a microbial cell of the disclosure comprises an exogenous polypeptide comprising an oxalate transporter selected from the group consisting of Lactobacillus acidophilusstrain La-14 WP_015613377, Bifidobacterium animalis subsp. Lactis BAH23805, and Oxalobacter formigenes Oxc.

In some embodiments. the engineered microbial cell provided herein comprises at least one exogenous polypeptide comprising a oxalate transporter selected from the group consisting of Lactobacillus acidophilus strain La-14 WP_015613377, Bifidobacterium animalis subsp. Lactis BAH23805, and Oxalobacter formigenes Oxc or a variant thereof. In some embodiments, the oxalate transporter is derived from or is a microbial oxalate transporter.

In some preferred embodiments, the microbial cell of the disclosure comprises an exogenous polypeptide comprising a oxalate transporter selected from those set forth in Table 2. below. comprising or consisting of the amino acid sequence of any one of SEQ ID NO: [symbol] , SEQ ID NO: [symbol] , SEQ ID NO: [symbol] , or a variant thereof. In some embodiments, the oxalate transporter comprises a Lactobacillus acidophilus strain La-14 WP_015613377 comprising the amino acid sequence set forth in SEQ ID NO: [symbol] . In some embodiments, the oxalate transporter comprises a Bifidobacterium animalis subsp. Lactis BAH23805 comprising the amino acid sequence set forth in SEQ ID NO: [symbol] . In some embodiments, the oxalate transporter comprises an Oxalobacter formigenes oxalate:formate antiporter comprising the amino acid sequence set forth in SEQ ID NO: [symbol] .

In some embodiments. the oxalate transporter comprises a variant of a wild-type oxalate transporter having at least 60% sequence identity to the amino acid sequence of any one of SEQ ID NO: [symbol] , SEQ ID NO: [symbol] , or SEQ ID NO: [symbol] . In some embodiment the variant of the oxalate transporter possesses a function of the wild-type oxalate transporter from which it was derived (e.g., the ability to transport oxalate). In a particular embodiment, the oxalate transporter consists of the amino acid sequence of any one of SEQ ID NO: [symbol] , SEQ ID NO: [symbol] , or SEQ ID NO: [symbol] . In general, a variant oxalate transporter, may be produced. for example, to enhance production of the protein in an engineered cell. to improve turnover/half-life of the protein or mRNA encoding the protein, and/or to modulate (enhance or reduce) the activity of the oxalate transporter. The oxalate transporter may also be in a form that is truncated, either at the amino terminal, or at the carboxyl terminal, or at both terminals.

In some embodiments, the invention provides an engineered microbial cell (e.g. an engineered bacterial cell) comprising a nucleic acid sequence encoding an oxalate transporter as described herein. In some embodiments, the invention provides an engineered microbial cell prepared by using a nucleic acid sequence encoding a oxalate transporter as described herein. In some embodiments, the nucleic acid sequence encodes an oxalate transporter (Lactobacillus acidophilus strain La-14 WP_015613377, Bifidobacterium animalis subsp. Lactis BAH23805, and Oxalobacter formigenes Oxc) as described herein.

### Polypeptides and Nucleic Acids

In one aspect, the disclosure provides isolated oxalate catabolismpolypeptides (e.g., oxalate decarboxylase) and oxalate transporters described herein. In some embodiments, the oxalate catabolismopolypeptides comprise an amino acid sequence having at least 60% sequence identity to the amino acid sequences of an oxalate catabolismpolypeptide described herein. In some embodiments, the oxalate transporters comprise an amino acid sequence having at least 60% sequence identity to the amino acid sequences of an oxalate transporter described herein. In some embodiments, the oxalate catabolismpolypeptides and oxalate transporters are engineered. Methods for producing engineered proteins are known in the art and described herein.

In another aspect. the disclosure provides nucleic acids (e.g., DNA or RNA (e.g., mRNA)) encoding a oxalate catabolismpolypeptide described herein. In another aspect, the disclosure provides nucleic acids (e.g., DNA or RNA (e.g., mRNA)) encoding an oxalate transporter described herein. In some embodiments, the nucleic acids are codon-optimized for expression in a desired cell type (e.g., a bacterial or fungal or archaeal cell).

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression, activity, stability, or other desirable parameters. Populations of Engineered Microbial Cells

In one aspect, the invention features cell populations comprising the engineered microbial cells of the invention, e.g., a plurality or population of the microbial cells. In various embodiments, the engineered microbial cell population comprises predominantly bacterial cells.

It will be understood that during the preparation of the engineered microbial cells of the invention, some fraction of cells may not contain the exogenous polypeptide or be transformed to express an exogenous polypeptide. Accordingly, in some embodiments, a population of engineered microbial cells provided herein comprises a mixture of engineered microbial cells and unmodified microbial cells, i.e., some fraction of cells in the population will not comprise, present, or express an exogenous polypeptide.

### Oxalate Consumption Assays

Quantitative oxalate consumption assays may be used to select cells for use in treating conditions in which oxalate is detrimental. Oxalate consumption assays can be used to quantify uptake from and/or biodegradation of oxalate from media surrounding intact cells. Oxalate consumption assays may be used to select cells suitable for treating conditions in which oxalate is detrimental following identification cells with improved oxalate degradation.

Oxalate consumption assays may comprise the steps of 1) creating an assay solution including an assay buffer comprising oxalate; 2) said assay buffer being substantially similar in pH to the pH of the intestinal lumen; 3) washing and suspending a known amount of cells in said assay buffer; 4) incubating the cells in said assay buffer under conditions suitable for oxalate degradation; 5) sampling aliquots of the assay solution at intervals; 6) measuring the formate concentration resulting from oxalate breakdown in the aliquots; and 7) selecting cells producing higher levels of formate for use in treating conditions in which oxalate is detrimental.

### Methods of Obtaining Engineered Microbial Cells

Various methods of obtaining genetically engineered microbial cells, e.g., bacterial cells, are contemplated by the present disclosure.

Methods of manufacturing microbial cells comprising an exogenous agent (e.g., a polypeptide) are described, e.g., in Yeast Protocols Handbook, Clontech Laboratories, Mountain View, USA, 2009.

In one aspect, the disclosure features an engineered microbial cell (e.g., engineered bacterial cell), comprising a first exogenous polypeptide comprising a oxalate catabolismpolypeptide, or a variant thereof, produced by a process comprising introducing an exogenous nucleic acid encoding the first exogenous polypeptide into a microbial cell; and culturing the microbial cell under conditions suitable for production of the first exogenous polypeptide.

In another aspect, the disclosure features an engineered microbial cell (e.g., engineered bacterial cell), comprising a first exogenous polypeptide comprising a oxalate transporter, or a variant thereof, produced by a process comprising introducing an exogenous nucleic acid encoding the first exogenous polypeptide into a microbial cell; and culturing the microbial cell under conditions suitable for production of the first exogenous polypeptide.

In another aspect, the disclosure features an engineered microbial cell (e.g., engineered bacterial cell), comprising a first exogenous polypeptide comprising an oxalate catabolism polypeptide, or a variant thereof, and a second exogenous polypeptide comprising an oxalate transporter, or a variant thereof, produced by a process comprising introducing an exogenous nucleic acid encoding the first exogenous polypeptide into a microbial cell; introducing an exogenous nucleic acid encoding the second exogenous polypeptide into a microbial cell; and culturing the microbial cell under conditions suitable for production of the first exogenous polypeptide and the second exogenous polypeptide.

In some embodiments, the oxalate catabolismpolypeptide is an oxalate decarboxylase, or a variant thereof. In some embodiments, more than one oxalate catabolismpolypeptide, or variant thereof, may be combined in one or more microbial cells, as described herein.

The processes of making the engineered microbial cells are described in more detail below.

### Probiotic Cells

Provided herein are engineered microbial cells, and methods of making the engineered microbial cells.

As used herein, the term “probiotic” refers to a live microbial cells that, when administered in adequate amounts, may or may not confer a health benefit on the host, and do not exert harmful effects on the host. Probiotic cells may be referred to or sold using alternative designations, for example as “nutraceuticals”, “dietary supplements”, “supplements”, “food additives”, “dietary ingredients”, “food ingredients”, and “ingredients”.

The engineered microbial cells can be probiotic cells. The engineered probiotic cells can be eukaryotic, e.g., fungal, e.g. Saccharomyces boulardii, e.g., Candida utilis, e.g. from the genus Kluyveromyces, microbial, e.g. from the genus Lactobacillus, or can be archeal. The engineered microbial cell can be from the genus Escherichia, e.g., Escherichia coli Nissle. The engineered microbial cell can be from the genus Bacteroides, e.g., Bacteroides ovatus, Bacteroides fragilis, Bacteroides thetaiotaomicron, Bacteroides vulgatus, Bacteroides ovatus, or Bacteroides uniformis.The engineered microbial cell can be from the genus Clostridium. The engineered microbial cell can be from the genus Bacillus (e.g. Bacillus subtilis).

### Expression of Exogenous Polypeptides

In some embodiments, the engineered microbial cells described herein are generated by contacting a suitable isolated cell, e.g., a microbial cell, with an exogenous nucleic acid encoding a polypeptide of the disclosure (e.g., an oxalate decarboxylase and/or a oxalate transporter).

In some embodiments, the exogenous polypeptide is encoded by a DNA, which is contacted with a microbial cell. In some embodiments, the exogenous polypeptide is encoded by an RNA, which is contacted with a microbial cell.

An exogenous polypeptide may be expressed from a transgene introduced into an microbial cell, e.g. by electroporation, chemical or polymeric transfection; an exogenous polypeptide that is over-expressed from the native locus by the introduction of an external factor, e.g., a transcriptional activator, transcriptional repressor, or secretory pathway enhancer.

In certain embodiments, the introducing step comprises electroporation. In some embodiments, the introducing step comprises chemical transformation (e.g., PEG-mediated transformation).

In some embodiments, the introducing step comprises introducing the first exogenous nucleic acid encoding the first exogenous polypeptide by electroporation of an episomal plasmid.

Exogenous polypeptides (e.g., an oxalate decarboxylase or a oxalate transporter) can be introduced by transfection of single or multiple copies of genes, transformation, or electroporation in the presence of DNA or RNA. Methods for expression of exogenous proteins in microbial cells are well known in the art.

In some embodiments, when there is more than one polypeptide (e.g., two or more), the polypeptides may be encoded in a single nucleic acid, e.g., a single vector. When both the oxalate decarboxylase and oxalate transporter are encoded in the same vector, there are multiple possible sub-strategies useful for this method of co-expression. In some embodiments, the single vector has a separate promoter for each gene, or any other suitable configuration. In some embodiments, the engineered nucleic acid comprises a gene encoding a first exogenous polypeptide, wherein the first exogenous polypeptide is an oxalate decarboxylase, or a variant thereof, and a gene encoding a second exogenous polypeptide, wherein the second exogenous polypeptide is an oxalate transporter, or a variant thereof.

For dual expression via 2 promoters in Bacillus subtilis, the NBP3510 promoter may be used as promoter #1 and as promoter #2, although the disclosure is not to be limited by this exemplary promoter. Another strategy is to express both oxalate decarboxylase and oxalate transporter proteins by inserting an internal ribosome entry site (IRES) between the two genes. Still another strategy is to express oxalate decarboxylase and oxalate transporter as direct peptide fusions separated by a linker.

In some embodiments, the two or more polypeptides are encoded in two or more nucleic acids, e.g., each vector encodes one of the polypeptides.

In certain embodiments, the expression vector comprises a promoter selected from the group consisting of Saccharomyces PDC1p, FBA1p, TEF2p, PGK1p, PGI1p, ADH1p, TDH2p, PYK1p, ENO2p, GPDp, GPM1p, TPI1p, TEF1p, and HXT7p promoters, as described in Sun et al, Biotechnology and Bioengineering 109:2082-2092 (2012).

Nucleic acids such as DNA expression vectors or mRNA for producing the exogenous polypeptides may be introduced into progenitor cells that are suitable to produce the exogenous polypeptides described herein. In some instances, the expression vectors can be designed such that they can incorporate into the genome of cells by homologous or non-homologous recombination by methods known in the art.

According to some embodiments, one or more exogenous polypeptides may be cloned into plasmid constructs for transfection. Methods for transferring expression vectors or genes into cells that are suitable to produce the engineered microbial cells described herein include, but are not limited to, transformation, chemical or polymeric transformation.

According to some embodiments, engineered DNA encoding each exogenous polypeptide may be cloned into a suitable integrative plasmid for integration into microbial cells. In some embodiments, the episomal or integrative vector comprises DNA encoding a single exogenous polypeptide for integration into microbial genomes. For example, in some embodiments, the episomal or integrative vector comprises DNA that will following integration result in microbial cells encoding an oxalate decarboxylase polypeptide under control of a non-inducible promoter. In some embodiments, the episomal or integrative vector comprises DNA encoding an oxalate transporter for integration into microbial cells. In other embodiments, the episomal or integrative vector comprises two, three, four or more exogenous polypeptides as described herein for integration into microbial cells. For example, in some embodiments, the episomal or integrative vector comprises DNA encoding an oxalate decarboxylase polypeptide and an oxalate transporter polypeptide for integration into microbial cells. According to some embodiments, engineered DNA encoding the one or more exogenous polypeptides may be cloned into a plasmid DNA construct encoding a selectable trait, such as an antibiotic resistance gene or an auxotrophy complementation gene. According to some embodiments, engineered DNA encoding the exogenous polypeptides may be cloned into a plasmid construct that is adapted to stably express each engineered protein in the microbial cells.

In some embodiments, the engineered microbial cell is generated by contacting a suitable isolated microbial precursor cell with an exogenous nucleic acid encoding one or more exogenous polypeptides. In some embodiments, the exogenous polypeptide is encoded by a DNA, which is contacted with a microbial precursor cell.

The one or more exogenous polypeptides may be genetically introduced into a microbial cell (e.g., Bacterial cell), using a variety of DNA techniques, including transient or stable transfections and gene transfer approaches. The exogenous polypeptides may be expressed on the surface and/or in the cytoplasm and/or in other subcellular compartments of the engineered microbial cells.

Optionally, electroporation methods may be used to introduce a plasmid vector into suitable microbial cells. Electroporation allows for the introduction of various molecules into the cells including, for example, DNA and RNA. As such, microbial cells are isolated and cultured as described herein.

Electroporation may be done using, for example, a MicroPulser Electroporator or Gene Pulser (Bio-Rad), as described in Benatuil et al, Protein Eng Des Sel. 23:155-159 (2010), Supplementary Methods.

Microbial cells may be transformed with an integrative expression vector which is unable to self-replicate. Alternatively, microbial cells may be transformed with a vector which may persist as autonomously replicating genetic units without integration into chromosomes. These vectors (e.g., plasmids) may exploit genetic elements derived from plasmids that are normally extrachromosomally replicating in cells. Such plasmids include, for example, the pUC19 (replicates in E. coli) and pTA1015 (replicates in B. subtilis) plasmid. Self-replicating vectors may also include chromosomal elements that allow for independent replication. Such self-replicating vectors exploit the cell's endogenous replication and chromosome segregation machinery to persist like mini-chromosomes. Chromosomal elements that can be used to produce self-replicating vectors include, for example, an autonomously replicating sequence (ARS) and a centromere (CEN), as described for example in Gnügge and Rudolf, Yeast 34:205-221 (2017).

Exogenous nucleic acids encoding one or more exogenous polypeptides may be assembled into expression vectors by standard molecular biology methods known in the art, e.g., restriction digestion, overlap-extension PCR, and Gibson assembly.

In certain embodiments, the engineered microbial cell is a microbial cell that presents a first exogenous polypeptide that is conjugated with a second exogenous polypeptide.

### Methods of Use and Treatment

The present disclosure provides methods of treating or preventing conditions in which oxalate is detrimental in a subject, comprising administering to the subject the engineered microbial cell as described herein, in an amount effective to treat or prevent conditions in which oxalate is detrimental in the subject.

Methods of administering engineered microbial cells comprising (e.g., presenting) exogenous agents (e.g., polypeptides) are described, e.g., in Govender et al, Aaps PharmSciTech 15:29-43 (2014).

In embodiments, the engineered microbial cells described herein (e.g., engineered bacterial cells) are orally administered to a subject, e.g., a mammal, e.g., a human. The methods described herein are applicable to both human therapy and veterinary applications.

In one aspect, the present disclosure provides a method of treating or preventing conditions in which oxalate is detrimental in a subject, comprising orally administering to the subject an engineered microbial cell as described herein (e.g. an engineered microbial cell comprising an oxalate catabolismpolypeptide, e.g., oxalate decarboxylase, an engineered microbial cell comprising a oxalate transporter, an engineered microbial cell comprising a oxalate catabolismpolypeptide, e.g., oxalate decarboxylase and an oxalate transporter), in an amount effective to treat or prevent excessive oxalate levels in the subject.

Dietary oxalate is plant-derived and may be a component of vegetables, nuts, fruits, and grains. In normal individuals, approximately half of urinary oxalate is derived from the diet and half from endogenous synthesis. The amount of oxalate excreted in urine plays an important role in for example calcium oxalate stone formation. Healthy individuals normally excrete urinary oxalate in ranges between 20-40 mg of oxalate per 24 hours. Urinary oxalate excretion at concentrations exceeding 40-45 mg per 24 hours is clinically considered hyperoxaluria (Robijn, Hoppe, Vervaet, D'Haese, & Verhulst, 2011). Hyperoxaluria is characterized by increased urinary excretion of and elevated systemic levels of oxalate. What may be in the normal range for the population as a whole may be elevated for an individual. Individuals with oxalate excretions >25 mg/day may benefit from a reduction of urinary oxalate output. The 24-h urine assessment may miss periods of transient surges in urinary oxalate excretion, which may promote stone growth and is a limitation of this analysis. If left untreated, hyperoxaluria can cause significant morbidity and mortality, including the development of renal stones (kidney stones), nephrocalcinosis (increased calcium in the kidney) and most significantly, End Stage Renal Disease

Cardiovascular and other consequences of elevated oxalate levels can occur with daily excretion levels well within these “normal” ranges. Therefore, a diagnosis of hyperoxaluria is not necessarily a prerequisite for the beneficial effects of the engineered microbial cells of the invention.

In some embodiments, the subject excretes more than 25 mg per day of oxalate prior to administering the engineered microbial cell.

In some embodiments, the methods described herein comprise selecting a subject excreting more than 25 mg of oxalate per day, and administering an engineered microbial cell described herein.

In some embodiments, the subject excretes less than about 40 mg per day after administering the engineered microbial cell.

### Conditions in Which Oxalate is Detrimental

An engineered microbial cell as described herein (e.g. an engineered microbial cell comprising a oxalate catabolismpolypeptide, e.g., oxalate decarboxylase, an engineered microbial cell comprising a oxalate transporter, or an engineered microbial cell comprising a oxalate catabolism polypeptide, e.g., oxalate decarboxylase and a oxalate transporter), alone or in combination with another agent, e.g., another agent described herein, can be used to treat, e.g., Hyperoxaluria, Hyperoxalemia, Nephrocalcinosis, Nephrolithiasis, and Urinary Tract Stones, or another conditiond associated with elevated urinary oxalate concentrations.

### Dosing

In one embodiment, a dose of engineered microbial cells as described herein comprises about 10\hat{ }6-10\hat{ }12 engineered microbial cells per dose.

In one example, administration of the engineered microbial cell is initiated at a dose which is minimally effective, and the dose is increased over a pre-selected time course until a positive effect is observed. Subsequently, incremental increases in dosage are made limiting to levels that produce a corresponding increase in effect while taking into account any adverse effects that may appear.

Any one of the doses provided herein for an engineered microbial cell as described herein can be used in any one of the methods or kits provided herein. Generally, when referring to a dose to be administered to a subject the dose is a label dose. Thus, in any one of the methods provided herein the dose(s) are label dose(s).

Also provided herein are a number of possible dosing schedules. Accordingly, any one of the subjects provided herein may be treated according to any one of the dosing schedules provided herein. As an example, any one of the subject provided herein may be treated with an engineered microbial cell as described herein. In certain embodiments, the engineered microbial cell comprises a first exogenous polypeptide comprising oxalate decarboxylase, or a variant thereof, and a second exogenous polypeptide comprising a oxalate transporter, or a variant thereof.

Each dose of engineered microbial cells can be administered at intervals such as thrice, twice, or once daily, once weekly, twice weekly, once monthly, or twice monthly. In some embodiments, a subject is dosed on a monthly dosing schedule.

The mode of administration for the composition(s) of any one of the treatment methods provided may be by oral administration, such as a capsule containing (freeze-)dried microbes, a powder containing (freeze-)dried microbes, or a suspension containing live microbes, prior to, during, or after a meal. Additionally, any one of the methods of treatment provided herein may also include administration of an additional therapeutic, as described in more detail below. The administration of the additional therapeutic may be according to any one of the applicable treatment regimens provided herein.

In some embodiments of any one of the methods provided herein, the level of oxalate is measured in the subject at one or more time points before, during and/or after the treatment period.

The methods described herein are intended for use with any subject that may experience the benefits of these methods. Thus, “recipients” “subjects.” “patients,” and “individuals” (used interchangeably) include humans as well as non-human subjects, particularly domesticated animals. Subjects provided herein can be in need of treatment according to any one of the methods or compositions or kits provided herein. Such subjects include those with elevated serum oxalate levels or oxalate deposits. Such subjects include those with hyperoxaluria or hyperoxalemia. It is within the skill of a clinician to be able to determine subjects in need of a treatment as provided herein.

In some embodiments, the subject and/or animal is a mammal, e g., a human. In some embodiments, the human is a pediatric human. In other embodiments, the human is an adult human. In other embodiments, the human is a geriatric human. In other embodiments, the human may be referred to as a patient.

In other embodiments, the subject is a non-human animal, and therefore the disclosure pertains to veterinary use. In a specific embodiment, the non-human animal is a household pet. In another specific embodiment, the non-human animal is a livestock animal.

In some embodiments, any one of the subjects for treatment as provided in any one of the methods provided has a condition associated with oxalate or another condition as provided herein. In some embodiments, any one of the subjects for treatment as provided in any one of the methods provided has been diagnosed with a disease selected from the group consisting of In one embodiment the disorder in which oxalate is detrimental is a disorder or disease selected from the group consisting of: PHI, PHII, PHII, secondary hyperoxaluria, enteric hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, hyperoxalemia, calcium oxalate nephrocalcinosis, calcium oxalate nephrolithiasis, and calcium oxalate urolithiasis.

In some embodiments, the subject has or is at risk of having an elevated oxalate level, e.g., an elevated plasma or serum oxalate level. When levels of oxalate exceed the physiologic limit of solubility, calcium oxalate may crystallize, and may cause calcium oxalate nephrocalcinosis, calcium oxalate nephrolithiasis, calcium oxalate urolithiasis, and other oxalate-associated conditions.

In some embodiments, daily urinary oxalate excretion in ranges between 20-40 mg of oxalate per 24 hours, or exceeding 40-45 mg per 24 hours may be indicative that a subject may be a candidate for treatment with any one of the methods or compositions or kits described herein.

In some embodiments, the subject has, or is at risk of having, calcium oxalate nephrocalcinosis, calcium oxalate nephrolithiasis, calcium oxalate urolithiasis, or other oxalate-associated conditions. In some embodiments, the subject has, or is at risk of having, PHI, PHII, PHII, secondary hyperoxaluria, enteric hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, hyperoxalemia, calcium oxalate nephrocalcinosis, calcium oxalate nephrolithiasis, and calcium oxalate urolithiasis

In some embodiments, the subject is selected for treatment with an microbial cell engineered to degrade oxalate of the present disclosure. In some embodiments, the subject is selected for treatment of hyperoxaluria with an engineered microbial cell of the present disclosure. In some embodiments, the subject is selected for treatment of hyperoxalemia with an engineered microbial cell of the present disclosure. In some embodiments, the subject is selected for treatment of calcium oxalate nephrocalcinosis with an engineered microbial cell of the present disclosure. In some embodiments, the subject is selected for treatment of calcium oxalate nephrolithiasis with an engineered microbial cell of the present disclosure. In some embodiments, the subject is selected for treatment of calcium oxalate urolithiasis with an engineered microbial cell of the present disclosure.

In certain embodiments, the methods of the present disclosure provide treatment of diseases or disorders associated with conditions in which oxalate is detrimental to human patients suffering therefrom. The treatment population is thus human subjects diagnosed as suffering from or at risk of suffering from hyperoxalemia, hyperoxaluria, calcium oxalate nephrocalcinosis, calcium oxalate nephrolithiasis, and calcium oxalate urolithiasis.

### Pharmaceutical Compositions

The present disclosure encompasses the preparation and use of pharmaceutical compositions comprising an engineered microbial cell (e.g., engineered bacterial cells) of the disclosure as an active ingredient. Such a pharmaceutical composition may consist of the active ingredient alone, as a combination of at least one active ingredient (e.g., an effective dose of an engineered bacterial cell) in a form suitable for administration to a subject, or the pharmaceutical composition may comprise the active ingredient and one or more pharmaceutically acceptable carriers, one or more additional (active and/or inactive) ingredients, or some combination of these.

In some embodiments, a pharmaceutical composition comprises a plurality of the engineered bacterial cells described herein, and a pharmaceutically acceptable carrier. In further embodiments, the pharmaceutical composition comprises a therapeutically effective dose of the engineered microbial cells.

In some embodiments, the pharmaceutical composition comprises between 10\hat{ }6 and 10\hat{ }12 engineered microbial cells.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the pharmaceutical compositions may be carried out in any convenient manner, including by aerosol inhalation, injection, ingestion, transfusion, implantation or transplantation. The compositions of the present disclosure may be administered to a patient orally.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for oral, or another route of administration.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

The pharmaceutical compositions may be prepared, packaged, or sold in the form of a a capsule or pill containing (freeze-)dried or metabolically active engineered microbes, a powder containing (freeze-)dried or metabolically active engineered microbes, or a suspension containing (freeze-)dried or metabolically active live microbes. These solids or liquids may be formulated according to the known art, and may comprise, in addition to the active ingredient, additional ingredients such as the dispersing agents, wetting agents, or suspending agents described herein. Such formulations may be prepared using a non-toxic orally-acceptable substance, such as cellulose, for example.

Other acceptable substances include, but are not limited to, guar gum, hypromellose (hydroxypropyl methylcellulose), inulin, fructooligosaccharides, gelatin, magnesium stearate, Silicon dioxide, rice bran extract, and lactose.

Formulation methods are described by e.g., Martins et al, Letters in Applied Microbiology 49:738-744 (2009), and by Joshi and Thorat, Drying Technology, 29:749-757 (2011).

The engineered microbial cell of the disclosure can be administered to an animal, e.g., a human. Where the engineered microbial cell are administered, they can be administered in an amount ranging from about 10\hat{ }6 to about 10\hat{ }12 cells wherein the cells are administered to the animal, preferably, a human patient in need thereof. While the precise dosage administered will vary depending upon any number of factors, including but not limited to, the type of animal and type of disease state being treated, the age of the animal and the route of administration.

The engineered microbial cell may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An engineered microbial cell may be co-administered with the various other compounds (e.g. other therapeutic agents). Alternatively, the compound(s) may be administered in advance of or after administration of the engineered microbial cell. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the engineered microbial cell, and the like.

Provided herein are compositions that may be administered as pharmaceuticals, therapeutics, and/or cosmetics. One or more microorganisms described herein can be used to create a pharmaceutical formulation comprising an effective amount of the composition for treating a subject. The microorganisms can be in any formulation known in the art. Some non-limiting examples can include topical, capsule, pill, enema, liquid, injection, and the like. In some embodiments, the one or more strains disclosed herein may be included in a food or beverage product, cosmetic, or nutritional supplement.

In some embodiments, a pharmaceutical formulation as described herein comprises an enteric coating. The pharmaceutical formulation may be formulated as an enteric-coated pill. An enteric-coating can protect the contents of a formulation, for example, pill or capsule, from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers.

The enteric coating can be designed to dissolve at any suitable pH. In some embodiments, the enteric coating is designed to dissolve at a pH greater than about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

A composition can be substantially free of preservatives. In some applications, the composition may contain at least one preservative. In particular embodiments, pharmaceutical formulations as described herein may contain an effective amount of a preservative. An “effective” amount is any amount that preserves or increases the shelf life of the pharmaceutical formulation beyond what would be obtained if the preservative were not present in the formulation. Examples of such preservatives include, but are not limited to, Vitamin E. Vitamin C. butylatedhydroxyanisole (BHA), butylatedhydroxytoluene (BHT), disodium ethylenediaminetetraacetic acid (EDTA), polyphosphates, citric acid, benzoates. sodium benzoate, sorbates, propionets, and nitrites.

The formulation can include one or more active ingredients. Active ingredients include, but are not limited to, antibiotics, prebiotics, probiotics, glycans (e.g., as decoys that would limit specific bacterial/viral binding to the intestinal wall), bacteriophages, microorganisms, bacteria, and the like.

In some embodiments, the formulation comprises a prebiotic. In some embodiments, the prebiotic is inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose. β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof. The prebiotic can serve as an energy source for the microbial formulation.

A formulation can be formulated for administration by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract.

Pharmaceutical formulations can be formulated as a dietary supplement. Pharmaceutical formulations can be incorporated with vitamin supplements. pharmaceutical formulations can be formulated in a chewable form such as a probiotic gummy. Pharmaceutical formulations can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A formulation of the disclosure can be administered as part of a fecal transplant process. A formulation can be administered to a subject by a tube, for example, nasogastric tube, nasojejunal tube, nasoduodenal tube, oral gastric tube, oral jejunal tube, or oral duodenal tube. A formulation can be administered to a subject by colonoscopy, endoscopy, sigmoidoscopy, and/or enema.

In some embodiments, the pharmaceutical formulation is formulated such that the one or more microbes can replicate once they are delivered to the target habitat (e.g. the gut). In one non-limiting example, the microbial composition is formulated in a pill, such that the pill has a shelf life of at least 1, 2, 3, 4, 5, 6, 7, 8, 9, 10, 11, or 12 months. In another non-limiting example, the storage of the microbial composition is formulated so that the microbes can reproduce once they are in the gut. In some embodiments, other components may be added to aid in the shelf life of the microbial composition. In some embodiments, one or more microbes may be formulated in a manner that it is able to survive in a non-natural environment. For example, a microbe that is native to the gut may not survive in an oxygen-rich environment. To overcome this limitation, the microbe may be formulated in a pill that can reduce or eliminate the exposure to oxygen. Other strategies to enhance the shelf-life of microbes may include other microbes (e.g. if the composition comprises elements whereby one or more strains is helpful for the survival of one or more strains).

In some embodiments, one or more of the microbes are lyophilized (e.g., freeze-dried) and formulated as a powder, tablet, enteric-coated capsule (e.g. for delivery to ileum/colon), or pill that can be administered to a subject by any suitable route. The lyophilized formulation can be mixed with a saline or other solution prior to administration.

In some embodiments, a composition is formulated for oral administration, for example, as an enteric-coated capsule or pill. for delivery of the contents of the formulation to the ileum and/or colon regions of a subject.

In some embodiments, the composition is formulated for oral administration. In some embodiments, the composition is formulated as an enteric-coated pill or capsule for oral administration. In some embodiments, the composition is formulated for delivery of the microbes to the ileum region of a subject. In some embodiments, the composition is formulated for delivery of the microbes to the colon region (e.g. upper colon) of a subject. In some embodiments, the composition is formulated for delivery of the microbes to the ileum and colon regions of a subject.

In some embodiments, the administration of a formulation of the disclosure can be preceded by, for example, colon cleansing methods such as colon irrigation/hydrotherapy, enema, administration of laxatives, dietary supplements, dietary fiber, enzymes, and magnesium.

In some embodiments, the composition is formulated as a population of spores. Spore-containing formulations can be administered by any suitable route described herein. Orally administered spore-containing formulations can survive the low pH environment of the stomach. The amount of spores employed can be, for example, from about 1% w/w to about 99% w/w of the entire formulation.

Formulations provided herein can include the addition of one or more agents to the therapeutics or cosmetics in order to enhance stability and/or survival of microbes in the formulation. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL).

In some embodiments, a formulation comprises one or more recombinant microbes or microbes that have been genetically modified. In other embodiments, one or more microbes are not modified or recombinant. In some embodiments, the formulation comprises microbes that can be regulated, for example, a microbe comprising an operon or promoter to control microbial growth. Microbes as described herein can be produced, grown, or modified using any suitable methods, including recombinant methods.

A formulation can be customized for a subject. A custom formulation can comprise, for example, a prebiotic, a probiotic, an antibiotic, or a combination of active agents described herein. Data specific to the subject comprising for example age, gender, and weight can be combined with an analysis result to provide a therapeutic agent customized to the subject. For example, a subject's microbiome found to be low in a specific microbe relative to a sub-population of healthy subjects matched for age and gender can be provided with a therapeutic and/or cosmetic formulation comprising the specific microbe to match that of the sub-population of healthy subjects having the same age and gender as the subject.

Formulations provided herein can include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

A formulation can include carriers and/or excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, antioxidants, gums, coating agents, coloring agents, flavoring agents, dispersion enhancer, disintegrant, coating agents, plasticizers, preservatives, suspending agents, emulsifying agents, plant cellulosic material and spheronization agents, and any combination thereof.

Non-limiting examples of pharmaceutically-acceptable excipients can be found, for example. in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A pharmaceutical, therapeutic, or cosmetic composition can be encapsulated within a suitable vehicle, for example, a liposome, a microspheres, or a microparticle. Microspheres formed of polymers or proteins can be tailored for passage through the gastrointestinal tract directly into the blood stream. Alternatively, the compound can be incorporated and the microspheres, or composite of microspheres, and implanted for slow release over a period of time ranging from days to months.

A pharmaceutical, therapeutic, or cosmetic composition can be formulated as a sterile solution or suspension. The therapeutic or cosmetic compositions can be sterilized by conventional techniques or may be sterile filtered. The resulting aqueous solutions may be packaged for use as is, or lyophilized. The lyophilized preparation of the microbial composition can be packaged in a suitable form for oral administration, for example, capsule or pill.

The compositions can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compositions can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, can be used.

Pharmaceutical compositions can be formulated using one or more physiologically-acceptable carriers comprising excipients and auxiliaries, which facilitate processing of the microorganisms into preparations that can be used pharmaceutically. Formulation may be modified depending upon the route of administration chosen. Pharmaceutical compositions described herein may be manufactured in a conventional manner. for example, by means of conventional mixing, dissolving, granulating, vitrification, spray-drying, lyophilizing, dragee-making, levigating, encapsulating, entrapping, emulsifying or compression processes.

In some embodiments, the pharmaceutical formulation is manufactured in a dry form, for example, by spray-drying or lyophilization. In some embodiments, the formulation is prepared as a liquid capsule to maintain the liquid form of the microbes.

Compositions provided herein can be stored at any suitable temperature. The formulation can be stored in cold storage, for example, at a temperature of about −80° C., about −20° C., about −4° C., or about 4° C. The storage temperature can be, for example, about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° C. to about 8° C. Storage of microbial compositions at low temperatures, for example from about 2° C. to about 8° C., can keep the microbes alive and increase the efficiency of the composition, for example, when present in a liquid or gel formulation. Storage at freezing temperature, below 0° C., with a cryoprotectant can further extend stability.

The pH of the composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. If the pH is outside the range desired by the formulator, the pH can be adjusted by using sufficient pharmaceutically-acceptable acids and bases. In some embodiments, the pH of the composition is between about 4 and about 6.

Pharmaceutical compositions containing microbes described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Microbial compositions can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

In some embodiments, the pharmaceutical compositions provided herein comprise engineered (i.e. modified) microbial cells and unmodified microbial cells. For example, a single unit dose of microbial cells (e.g., modified and unmodified microbial cells) can comprise, in various embodiments, about, at least, or no more than 10%, 20%, 30%, 40%, 50%, 60%, 70%, 75%, 80%), 85%, 90%, 95%, or 99% engineered microbial cells, wherein the remaining microbial cells in the composition are not engineered.

In some embodiments of the above aspects and embodiments, the engineered microbial cell is a bacterial cell, e.g. Bacillus subtilis.

### Combination Therapies

According to some embodiments, the disclosure provides methods that further comprise administering an additional agent (e.g. an additional therapeutic) to a subject. In some embodiments, the disclosure pertains to co-administration and/or co-formulation.

Additional therapeutics for elevated oxalate levels, or conditions in which oxalate is detrimental, such as for example Hyperoxaluria, Hyperoxalemia, Nephrocalcinosis, Nephrolithiasis, and Urinary Tract Stones, may be administered to any one of the subjects provided herein, such as for the reduction of urinary and/or serum oxalate levels. Any one of the methods provided herein may include the administration of one or more of these additional therapeutics. In some embodiments, any one of the methods provided herein do not comprise the concomitant administration of an additional therapeutic. Examples of additional therapeutics include, but are not limited to, the following. Other examples will be known to those of skill in the art.

Alkalinizing agents such as potassium citrate are prescribed to decrease stone recurrence in patients with calcium nephrolithiasis. Citrate binds intestinal and urine calcium and increases urine pH.

Other examples of therapeutics are thiazide diuretics, which can reduce stone recurrence at least in part by reducing urine calcium loss and urine supersaturation. They act on the kidney but also seem to improve bone mineral balance and reduce fractures.

Further examples of additional therapeutics include but are not limited to other diuretics such as Acetazolamide.

Other additional therapies may comprise other probiotics, including for example non- engineered Oxalobacter formigenes, Bifidobacterium animalis, Lactobacillus acidophilus, or combinations thereof.

Additional therapeutics also include recombinant oxalate-catabolismenzyme based therapies, which may be modified for example by pegylation. Such therapies, such as when infused or when given orally, have been shown to reduce oxalate levels in the urine. ALLN-177 is an orally administered, oxalate-specific enzyme therapy to reduce urine oxalate (UOx) excretion in patients with secondary hyperoxaluria.

Any one of the methods provided herein, thus, can include the subsequent administration of an oral or other oxalate reducing therapeutic as an additional therapeutic subsequently or concurrently with the treatment regimen according to any one of the methods provided is performed.

The treatments provided herein may allow patients to subsequently or concurrently be treated with an oxalate lowering therapeutic, such as a diuretic.

Treatment according to any one of the methods provided herein may also include a pre-treatment with a therapeutic, such as with NSAIDS.

Monitoring of a subject, such as the measurement of serum or urine oxalate levels, may be an additional step further comprised in any one of the methods provided herein.

The following list of numbered embodiments is disclosed:

Embodiment 1: An engineered microbial cell, the microbial cell comprising: an exogenous nucleic acid encoding at least one oxalate catabolismenzyme.
Embodiment 2: The engineered microbial cell of embodiment 1, wherein the engineered microbial cell is a bacterial cell.
Embodiment 3: The engineered microbial cell of any of the preceding embodiments, wherein the encoded oxalate catabolismenzyme is constitutively expressed in the engineered microbial cell.
Embodiment 4: The engineered microbial cell of any of the preceding embodiments, wherein the at least one oxalate catabolismenzyme is an oxalate decarboxylase that directly catalyzes the reaction: oxalate+H⁺ formate+CO₂.
Embodiment 5: The engineered microbial cell of any of the preceding embodiments, wherein the at least one oxalate catabolismenzyme is a bacterial oxalate decarboxylase.
Embodiment 6: The engineered microbial cell of any of the preceding embodiments, wherein the at least one oxalate catabolismenzyme comprises at least 95% identity with the full length of any one of SEQ ID NOs: 1-4.
Embodiment 7: The engineered microbial cell of embodiment 5, wherein the oxalate decarboxylase is a mutant oxalate decarboxylase, a chimera, a and/or an overexpression variant of the oxalate decarboxylase.
Embodiment 8: A composition comprising:
the engineered microbial cell of any preceding embodiment, and
an enteric coating and/or a preservative.
   Embodiment 9: The composition of embodiment 8, wherein the composition is a pharmaceutical composition.
   Embodiment 10: The composition of embodiment 9, wherein the pharmaceutical composition comprises a pharmaceutically acceptable carrier.
   Embodiment 11: The composition of embodiment 9 or 10, wherein the pharmaceutical composition comprises an enteric coating and/or a preservative.
   Embodiment 12: A method of administering a microbial cell to a subject, the method comprising administering to the gastrointestinal tract of the subject the engineered microbial cell of any one of embodiments 1-7 or the composition of any one of embodiments 8-11.
   Embodiment 13: The method according to embodiment 12, wherein the administration of the engineered microbial cell lowers the level of oxalate in the gastrointestinal tract as compared to the level of level of oxalate in the gastrointestinal tract prior to administration.
   Embodiment 14: The method according to any one of embodiments 12 and 13, wherein the administration is oral administration.
   Embodiment 15: The method according to to any one of embodiments 12-14, wherein the subject suffers from a condition selected from the group consisting of: hyperoxaluria, hyperoxalemia, calcium oxalate nephrocalcinosis, calcium oxalate nephrolithiasis, calcium oxalate urolithiasis, and combinations thereof.
   Embodiment 16: The method according to to any one of embodiments 12 and 15, wherein administering the engineered microbial cell of claim 1 comprises administering 10\hat{ }6 to 10\hat{ }13 of the engineered microbial cells.
   Embodiment 17: The method according to to any one of embodiments 12 and 16, wherein administering the engineered microbial cell of claim 1 comprises administering about 10\hat{ }9 of the engineered microbial cells
   Embodiment 18: A method for selecting cells expressing extracellular oxalate decarboxylase the method comprising:
Suspending in an assay buffer comprising oxalate a known known number of microbial cells;
incubating the microbial cells in the assay buffer under conditions suitable for oxalate degradation;
measuring the formate concentration in the assay buffer; and
selecting microbial cells producing higher levels of formate.

### EXAMPLE 1

### Example 1: “Bacillus subtilis Strain PY79 Derivative Cells Genetically Engineered to Overexpress Endogenous OxdC”

Integrative shuttle vector for insertion of the NB3510 promoter upstream of the start codon of the endogenous oxdC gene in the Bacillus subtilis strain PY79 chromosome

An Integrative shuttle vector was designed, encoding two homologous sequences of 800 bps to facilitate targeted insertion into the Bacillus subtilis chromosome, 800 bps upstream of the start codon of the Bacillus subtilis oxde gene, and the first 800 bps of the Bacillus subtilis oxdC gene, with the 121 bps NPB3510 strong constitutive promoter between the homologous sequences. The sequence of the recombination sequence is set forth in SEQ ID NO: [symbol] .

The DNA of SEQ ID NO: [symbol] with flanking SbfI and BamHI restriction site sequences was obtained from Integrated DNA Technologies (Coralville, IA), and cloned into the corresponding SbfI and BamHI sites in the multiple cloning site of the pMiniMad2 plasmid, which is a shuttle vector that can replicate in E. coli cells as well as in Bacillus subtilis cells, as described in (Patrick & Kearns, 2008).

The vector was amplified in E. coli NEB5a cells (Cat no. C2987, New England Biolabs), and extracted using methods well known in the art using the Monarch kit (Cat no. T1010, New England Biolabs). Bacillus subtilis strain PY79 from the Bacillus Genetic Stock Center was used (strain number 1A747) for all manipulations. Bacteria were grown in LB medium (1% tryptone, 0.5% yeast extract, 0.5% sodium chloride, with addition of 1.5% agar for solid media. For MLS resistance selection, 1 μg/mL erythromycin and 25 μg/mL lincomycin were used. For transformation experiments, bacteria were grown in modified competence (MC) medium (100 mM phosphate buffer, 2% glucose, 3 mM trisodium citrate, 22 mg/L ferric ammonium citrate, 0.1% cascin hydrolysate, 0.16% glutamic acid. 3 mM magnesium sulfate).

Plasmid DNA was used as the DNA source for chromosomal modifications. A single colony of Bacillus strain PY79 was picked, and inoculated in 2 mL of MC medium in a 15 mL test tube. The culture was grown at 37° C. with shaking at 275 rmp for 4.5 hours, or approx. 1 hour after the end of the exponential growth phase. 400 uL of the culture was transferred to a new 15 mL test tube, and 1 μg of pMiniMAD plasmid containing the fragment from SEQ ID NO: [symbol] was added. The culture with DNA was returned to the 37° C. shaker for 1.5 hours. Cultures were plated on LB agar with 1 μg/mL crythromycin and 25 μg/mL lincomycin, and incubated overnight at 37° C. Isolated colonies were screened for mutant allele via PCR using primer pairs where one primer annealed to a region outside the insert, and the other annealed to a region inside the insert. If the insert had recombined into the chromosome at the expected locus, PCR products of the expected size were obtained. A positive colony was then inoculated in 3 mL LB broth without antibiotics. This culture was grown overnight at room temperature with shaking at 275 rpm. The overnight culture was plated at various dilutions on LB agar plates without antibiotics and grown at 37° C. overnight. Because of the lack of antibiotic selection, the plasmid is lost during overnight replication. Isolated colonies were duplicate streaked on LB plates with and without selection antibiotics (1 μg/mL crythromycin and 25 μg/mL lincomycin), and grown overnight at 37° C. Antibiotic sensitive colonies were screened again with the same primer pairs to identify strains with the mutant allele.

### EXAMPLE 2

### Example 2: “Bacillus subtilis Strain PY79 Derivative Cells Genetically Engineered to Overexpress Endogenous OxdD”

Integrative shuttle vector for insertion of the NB3510 promoter upstream of the start codon of the endogenous oxdD gene in the Bacillus subtilis strain PY79 chromosome

An Integrative shuttle vector was designed, encoding two homologous sequences of 800 bps to facilitate targeted insertion into the Bacillus subtilis chromosome, 800 bps upstream of the start codon of the Bacillus subtilis oxdD gene, and the first 800 bps of the Bacillus subtilis oxdD gene. with the 121 bps NPB3510 strong constitutive promoter inbetween. The sequence of the recombination sequence is set forth in SEQ ID NO: [symbol] . The DNA of SEQ ID NO: [symbol] with flanking SbfI and BamHI restriction site sequences was obtained from Integrated DNA Technologies (Coralville, IA), and cloned into the corresponding SbfI and BamHI sites in the multiple cloning site of the pMiniMad2 plasmid. A recombinant PY79 derivative Bacillus subtilis strain overexpressing endogenous oxdD under control of the NB3510 promoter was then obtained using the integrative pMiniMad2 shuttle vector with the recombination sequence set forth in SEQ ID NO: [symbol] using the method outlined in Example 1.

### EXAMPLE 3

### Example 3: “Bacillus subtilis Strain PY79 Derivative Cells Genetically Engineered to Overexpress Both Endogenous OxdC and OxdD”

The Bacillus subtilis strain PY79 derivative strain overexpressing endogenous OxdC, obtained as set forth in Example 1, was further modified to overexpress endogenous OxdD in the manner described in Example 2.

The resulting recombinant Bacillus subtilis strain PY79 derivative strain expresses both of its endogenous oxdC and oxdD gene products under control of the NB3510 promoter from the Bacillus subtilis strain PY79 chromosome

### EXAMPLE 4

### Example 4: “Bacillus subtilis subsp. inaquosorum Strain DE111® Derivative Cells Genetically Engineered to Overexpress Endogenous OxdC”

Integrative shuttle vector for insertion of the NB3510 promoter upstream of the start codon of the endogenous oxdC gene in the Bacillus subtilis subsp. inaquosorum strain DE111® chromosome

An Integrative shuttle vector was designed, encoding two homologous sequences of approx. 800 bps to facilitate targeted insertion into the Bacillus subtilis subsp. inaquosorum strain DE11/® chromosome, 800 bps upstream of the start codon of the endogenous oxdC gene, and the first 800 bps of the endogenous oxdC gene, with the 121 bps NPB3510 strong constitutive promoter between the homologous sequences. The sequence of the recombination sequence is set forth in SEQ ID NO: [symbol] .

The DNA of SEQ ID NO: [symbol] with flanking SbfI and BamHI restriction site sequences was obtained from Integrated DNA Technologies (Coralville, IA), and cloned into the corresponding SbfI and BamHI sites in the multiple cloning site of the pMiniMad2 plasmid. Bacillus subtilis subsp. inaquosorum strain DE111® was used for all manipulations in the same manner as described in Example 1, except for the following: 10 μg of plasmid DNA was added to a 50 mL conical bottom cell culture tube (bioreaction tube) containing 5 mL competent B. subtilis D cell suspension grown in Modified Competence medium (MC medium) at an OD600 of >1.5. Cells were then incubated at 37° C. for 2 h on a shaker incubator at 250 RPM. 5 mL of SOC medium (2% tryptone, 0.5% yeast extract, 10 mM NaCl, 2.5 mM KCl, 10 mM MgCl₂, 10 mM MgSO₄, and 20 mM glucose) was added to allow for cell recovery in order to allow antibiotic resistance proteins to be expressed before plating. Cultures were incubated at 37° C. for 1 hour on a shaker incubator at 250 RPM. A 1:10 serial dilution series for was prepared for to plate dilutions. Under sterile conditions, 100 μL of the previous dilution was added to 900 μL plain LB medium in a 1.7 mL eppendorf tube. 10⁻¹, 10⁻², and 10⁻³ dilutions were prepared. The remaining 9.9 mL of cell suspension was pelleted by centrifugation at 3716×G in a benchtop centrifuge for 3 minutes, the clarified supernatant was removed, and the cells resuspended by vortexing in 500 μL plain LB medium. The resuspended cells, and 500 μLs of the 10⁻¹, 10⁻², and 10⁻³ dilutions were then plated on 10 cm MLS LB agar plates. Further steps used to isolated recombinant clones were the same as in Example 1.

### EXAMPLE 5

### Example 5: “Bacillus subtilis subsp. inaquosorum Strain DE111® Derivative Cells Genetically Engineered to Overexpress Endogenous OxdD”

Integrative shuttle vector for insertion of the NB3510 promoter upstream of the start codon of the endogenous oxdD gene in the Bacillus subtilis subsp. inaquosorum strain DE111® chromosome

An Integrative shuttle vector was designed, encoding two homologous sequences of approx. 800 bps to facilitate targeted insertion into the Bacillus subtilis subsp. inaquosorum strain DE111® chromosome, 800 bps upstream of the start codon of the endogenous oxdD gene, and the first 796 bps of the endogenous oxdD gene, followed by a stop codon, with the 121 bps NPB3510 strong constitutive promoter inbetween. The sequence of the recombination sequence is set forth in SEQ ID NO: [symbol] .

The DNA of SEQ ID NO: [symbol] with flanking SbfI and BamHI restriction site sequences was obtained from Integrated DNA Technologies (Coralville, IA), and cloned into the corresponding SbfI and BamHI sites in the multiple cloning site of the pMiniMad2 plasmid. A recombinant Bacillus subtilis subsp. inaquosorum strain DE111® overexpressing its endogenous oxdD gene product under control of the NB3510 promoter was then obtained using the Integrative pMiniMad2 shuttle vector with the recombination sequence set forth in SEQ ID NO: [symbol] using the method outlined in Example 1, modified as outline Example 4.

### EXAMPLE 6

### Example 6: “Bacillus subtilis subsp. inaquosorum Strain DE111® Derivative Cells Genetically Engineered to Overexpress Endogenous OxdC and OxdD”

The Bacillus subtilis subsp. inaquosorum strain DE111® derivative strain overexpressing endogenous OxdC, obtained as set forth in Example 4, was further modified to overexpress endogenous OxdD in the manner described in Example 5.

The resulting recombinant Bacillus subtilis subsp. inaquosorum strain DE111® derivative strain overexpresses both oxdC and oxdD, each under control of an NB3510 promoter.

### EXAMPLE 7

### Example 7. “Lactobacillus acidophilus La-14 Cells Genetically Engineered to Overexpress Endogenous Lactobacillus acidophilus La-14 Oxalate Catabolic Genes and Oxalate Transporter”

The native promoter driving expression of the Lactobacillus acidophilus La-14 operon encoding oxalate catabolismenzymes (Oxc and Frc) is replaced by a strong constitutive promoter such as the Pgm and SlpA promoters as described in (Nguyen et al., 2019).

Various approaches and tools to obtain chromosomal genetic modifications have been described for Lactobacillus, eg in (Mills, 2001). One skilled in the art can easily use the approach described therein to insert a strong constitutive promoter such as the Pgm and SlpA promoter ahead of the operon encoding the fre gene (encoding the protein of SEQ ID NO: [symbol] ) and oxe gene (encoding the protein of SEQ ID NO: [symbol] ) as described in (Azcarate-Peril, Bruno-Bárcena, Hassan, & Klaenhammer, 2006), in order to drive strong constitutive expression in Lactobacillus acidophilus La-14.

The native promoter driving expression of the Lactobacillus acidophilus La-14 gene encoding an oxalate transporter such as WP_015613377 as described in SEQ ID NO: [symbol] is then replaced by a strong constitutive promoter such as the Pgm and SlpA promoters as described in (Nguyen et al., 2019) using the approach as described for Lactobacillus by (Mills, 2001) in order to drive strong constitutive expression in Lactobacillus acidophilus La-14.

### EXAMPLE 8

### Example 8. “Bifidobacterium animalis subsp. lactis Cells Genetically Engineered to Overexpress Endogenous Bifidobacterium animalis subsp. lactis Oxalate Catabolic Genes and Oxalate Transporter”

The native promoters driving expression of the Bifidobacterium animalis subsp. lactis genes encoding oxalate catabolismenzymes (Oxc and Frc) are replaced by a strong constitutive promoter such as the P919 promoter as described in (Wang, Kim, Park, & Ji, 2012)

Various approaches and tools to obtain chromosomal genetic modifications have been described for Bifidobacterium, eg a double crossover recombination strategy in (Castro-Bravo, Hidalgo-Cantabrana, Rodriguez-Carvajal, Ruas-Madiedo, & Margolles, 2017). One skilled in the art can easily use the approach described therein to insert a strong promoter such as the P919 promoter as described in (Wang et al., 2012) ahead of the genes encoding the fre gene (encoding the protein of SEQ ID NO: [symbol] , described in (Turroni et al., 2010) as ORF-4 [symbol] ) and oxc gene (encoding the protein of SEQ ID NO: [symbol] ) as described in (Turroni et al., 2010), in order to drive strong constitutive expression in Bifidobacterium animalis subsp. lactis.

The native promoter driving expression of the Bifidobacterium animalis subsp. lactis gene encoding an oxalate transporter such as described in SEQ ID NO: [symbol] (described in as (Turroni et al., 2010) as ORF-1) is then replaced by a strong constitutive promoter such as the P919 promoter as described in (Wang et al., 2012) using the double crossover recombination strategy described by (Castro-Bravo et al., 2017).

### EXAMPLE 9

### Example 9. In Vivo Oxalate Consumption by B. subtilis Overexpressing Endogenous Oxalate Decarboxylase

Frozen cultures of strains prepared described in Examples 1-4, as well as B. subtilis strains DE111® and PY79 were plated on antibiotic-free LB plates and incubated overnight at 37° C. The next day, single colonies were inoculated into 4 mL of LB medium in 14 mL disposable roundbottom polystyrene test tubes. Cultures were incubated overnight in an incubator at 37° C. shaking at 350 RPM.

200 μL of cell cultures were transferred to 1.7 mL Eppendorf tubes. Cells were pelleted by centrifugation at 20000×G for 2 minutes, and the supernatant was removed with a p1000 micro pipette. Cells were resuspended in 500 μL PBS. Cells were pelleted by centrifugation at 20000×G for 2 minutes, and the supernatant was carefully removed. Cells were resuspended in was in 100 μL PBS supplemented with 3 mM MgSO4 and 2 gr/L glucose. 15 μL of the well mixed resuspended cells was then transferred to a 700 μL Eppendorf tube.

The Sigma-Aldrich Oxalate Decarboxylase Activity Assay Kit (MAK214) was used to test Oxalate Decarboxylase Activity.

Enzymatic reaction mix was prepared by mixing 8 volumes of OXDC Assay Buffer I with 2 volumes of OXDC Substrate solution. OXDC Buffer Mix, was prepared by mixing 1 volume of OXDC Assay Buffer I with1 volume of OXDC Assay Buffer II. Development Reaction Mix was prepared by mixing 46 volumes of OXDC Buffer Mix with 2 volumes of OXDC Enzyme Mix and 2 volumes of OXDC Probe.

10 μL of the enzymatic reaction mix was added to the 700 μL Eppendorf tube with 15 μL cell suspension. Negative control reactions were set up with 15 μL buffer I instead.

The mixture was mixed well by brief vortexing, and tubes were incubated at 37° C. for 60 minutes. Next, 25 μL OXDC Assay Buffer II was added to the tube to stop the enzymatic reaction. The mixture was mixed well by brief vortexing, and cells were pelleted by centrifugation at 20000×G for 2 minutes.

40 μL of Development Reaction Mix was added to a well of a 96 well optically clear flat bottom plate, and 40 uL of the clarified stopped reaction was added to the development reaction, and the components were mixed. The lid was added to the plate, and incubated at 37° C. for 40 minutes. Absorbance at 450 nm was then read in a BioRad Model 550 spectrophotometer.

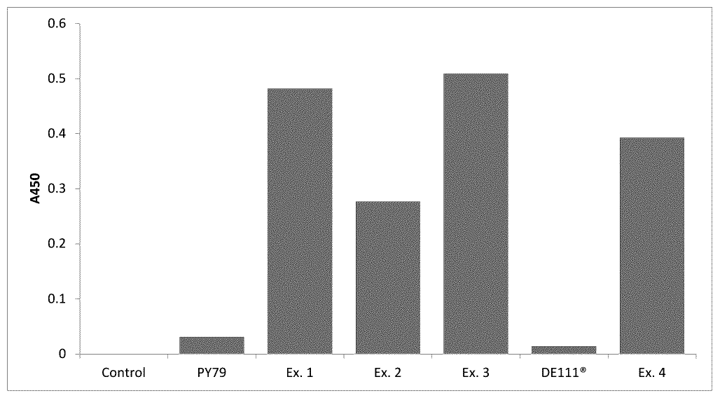

Reactions set up using a cell suspension of non-engineered B. subtilis strain PY79 and DE111 did not show a significant increase in A450. Reactions set up using B. subtilis cells engineered to overexpress oxdC, oxdD, or oxdC and oxdD as outlined in Examples 1-5 however showed a marked increase in A450, as is shown in FIG. 1.

Thus, we concluded intact B. subtilis cells overexpressing endogenous oxalate decarboxylase, but not non-engineered parental cells, were able to convert oxalate in a solution to highly soluble formate.

This was surprising, as B. subtilis oxalate decarboxylase is known to be an intracellular enzyme, as these cells were not engineered to overexpress oxalate and formate transporters or oxalate:formate antiporters, and as the assay detects the formation of formate, which is the product of the oxalate decarboxylase reaction.
Anantharam, V., Allison, M. J., & Maloney, P. C. (1989). Oxalate:formate exchange: The basis for energy coupling in Oxalobacter. Journal of Biological Chemistry, 264(13), 7244-7250. doi:https://doi.org/10.1016/S0021-9258(18)83227-6
Azcarate-Peril, M. A., Bruno-Bárcena, J. M., Hassan, H. M., & Klaenhammer, T. R. (2006). Transcriptional and functional analysis of oxalyl-coenzyme A (CoA) decarboxylase and formyl-CoA transferase genes from Lactobacillus acidophilus. Applied and Environmental Microbiology, 72(3), 1891-1899. doi:10.1128/aem.72.3.1891-1899.2006
Castro-Bravo, N., Hidalgo-Cantabrana, C., Rodriguez-Carvajal, M. A., Ruas-Madiedo, P., & Margolles, A. (2017). Gene Replacement and Fluorescent Labeling to Study the Functional Role of Exopolysaccharides in Bifidobacterium animalis subsp. lactis. Frontiers in Microbiology, 8(1405). doi:10.3389/fmicb.2017.01405
Hoppe, B., & Blau, N. (2014). Hyperoxalurias. In N. Blau, M. Duran, K. M. Gibson, & C. Dionisi Vici (Eds.), Physician's Guide to the Diagnosis, Treatment, and Follow-Up of Inherited Metabolic Diseases (pp. 465-474). Berlin, Heidelberg: Springer Berlin Heidelberg.
Iyalomhe, O., Khantwal, C. M., & Kang, D. C. (2015). The Structure and Function of OxIT, the Oxalate Transporter of Oxalobacter formigenes. J Membr Biol, 248(4), 641-650.
Mills, D. A. (2001). Mutagenesis in the post genomics era: tools for generating insertional mutations in the lactic acid bacteria. Curr Opin Biotechnol, 12(5), 503-509.
Monico, C. G., Persson, M., Ford, G. C., Rumsby, G., & Milliner, D. S. (2002). Potential mechanisms of marked hyperoxaluria not due to primary hyperoxaluria I or II. Kidney International, 62(2), 392-400. doi:https://doi.org/10.1046/j.1523-1755.2002.00468.x
Nguyen, H .- M., Pham, M .- L., Stelzer, E. M., Plattner, E., Grabherr, R., Mathiesen, G., . . . Nguyen, T.-H. (2019). Constitutive expression and cell-surface display of a bacterial β-mannanase in Lactobacillus plantarum. Microbial Cell Factories, 18(1), 76. doi:10.1186/s12934-019-1124-y
Patrick, J. E., & Kearns, D. B. (2008). MinJ (YvjD) is a topological determinant of cell division in Bacillus subtilis. Molecular Microbiology, 70(5), 1166-1179. doi:https://doi.org/10.1111/j.1365-2958.2008.06469.x
Robijn, S., Hoppe, B., Vervaet, B. A., D′Haese, P. C., & Verhulst, A. (2011). Hyperoxaluria: a gut-kidney axis? Kidney International, 80(11), 1146-1158. doi:10.1038/ki.2011.287
Turroni, S., Bendazzoli, C., Dipalo Samuele, C. F., Candela, M., Vitali, B., Gotti, R., & Brigidi, P. (2010). Oxalate-Degrading Activity in Bifidobacterium animalis subsp. lactis: Impact of Acidic Conditions on the Transcriptional Levels of the Oxalyl Coenzyme A (CoA) Decarboxylase and Formyl-CoA Transferase Genes. Applied and Environmental Microbiology, 76(16), 5609-5620. doi:10.1128/aem.00844-10
Wang, Y., Kim, J. Y., Park, M. S., & Ji, G. E. (2012). Novel Bifidobacterium promoters selected through microarray analysis lead to constitutive high-level gene expression. Journal of Microbiology, 50(4), 638-643. doi:10.1007/s12275-012-1591-x
Witting, C., Langman, C. B., Assimos, D., Baum, M. A., Kausz, A., Milliner, D., . . . Lieske, J. C. (2021). Pathophysiology and Treatment of Enteric Hyperoxaluria. Clinical Journal of the American Society of Nephrology, 16(3), 487-495. doi:10.2215/cjn.08000520

### SEQUENCES

TABLE 1
Exemplary Oxalate decarboxylases

| SEQ | | |
|---|---|---|
| ID | | |
| NO | Name | Amino acid sequence |
| 1 | Bacillus | MKKQNDIPQPIRGDKGATVKIPRNIERDRQNPDMLVPPETDHGTVSNMKFSFSDTHNRLE |
| | subtilis strain | KGGYAREVTVRELPISENLASVNMRLKPGAIRELHWHKEAEWAYMIYGSARVTIVDEKGR |
| | PY79 OxdC | SFIDDVGEGDLWYFPSGLPHSIQALEEGAEFLLVFDDGSFSENSTFQLTDWLAHTPKEVI |
| | | AANFGVTKEEISNLPGKEKYIFENQLPGSLKDDIVEGPNGEVPYPFTYRLLEQEPIESEG |
| | | GKVYIADSTNFKVSKTIASALVIVEPGAMRELHWHPNTHEWQYYISGKARMTVFASDGHA |
| | | RTFNYQAGDVGYVPFAMGHYVENIGDEPLVFLEIFKDDHYADVSLNQWLAMLPETFVQAH |
| | | LDLGKDFTDVLSKEKHPVVKKKCSK |
| 2 | Bacillus | MLLEQQPINHEDRNVPQPIRSDGAGAIDTGPRNIIRDIQNPNIFVPPVTDEGMIPNLRFS |
| | subtilis strain | FSDAPMKLDHGGWSREITVRQLPISTAIAGVNMSLTAGGVRELHWHKQAEWAYMLLGRAR |
| | PY79 OxdD | ITAVDQDGRNFIADVGPGDLWYFPAGIPHSIQGLEHCEFLLVFDDGNFSEFSTLTISDWL |
| | | AHTPKDVLSANFGVPENAFNSLPSEQVYIYQGNVPGSVASEDIQSPYGKVPMTFKHELLN |
| | | QPPIQMPGGSVRIVDSSNFPISKTIAAALVQIEPGAMRELHWHPNSDEWQYYLTGQGRMT |
| | | VFIGNGTARTFDYRAGDVGYVPSNAGHYIQNTGTETLWFLEMFKSNRYADVSLNQWMALT |
| | | PKELVQSNLNAGSVMLDSLRKKKVPVVKYPGT |
| 3 | Bacillus | MKKQNDIPQPIRGDKGATVKIPRNIERDRQNPDMLVPPETDHGTVSNMKFSFSDTHNRLE |
| | subtilis subsp. | KGGYAREVTVRELPISENLASVNMRLKPGAIRELHWHKEAEWAYMIYGSARVTIVDEKGR |
| | inaquosorum | SFIDDVGEGDLWYFPSGLPHSIQALDEGAEFLLVFDDGSFSENSTFQLTDWLAHTPKEVI |
| | strain | AANFGVTKEEIANLPGKEKYIFENQIPGSLKDDIVEGPNGEVPYPFTYRLLEQEPIESEG |
| | DE111 ® | GKVYIADSTNFTVSKTIASALVTVEPGAMRELHWHPNTHEWQYYISGKARMTVFASDGHA |
| | OxdC | RTFNYQAGDVGYVPFAMGHYVENIGDEPLVFLEIFKDDHYADVSLNQWLAMLPEKFVQAH |
| | | LDLGKDFTDVLSKEKHPVVKKKCSK |
| 4 | Bacillus | MEKQPINHEDRNVPQPIRSDGAGAIDAGPRNMMRDIQNPNILVPPVTDEGMIPNLRFSFS |
| | subtilis subsp. | DAPMKLDHGGWSREITVRQLPISTAIAGVNMSLTAGGVRELHWHKQAEWAYMLLGRARIT |
| | inaquosorum | AVDQEGRNFIADVGPGDLWYFPAGIPHSIQGLEHCEFLLVFDDGNFSEFSTLTISDWLAH |
| | strain | TPKDVLSGNFGVPENAFHSLPSEQVYIYQGNVPGSVASEDIQSPYGKVPLTFKYELLNQT |
| | DE111 ® | PIQTPGGSVRIVDSSNFPISKNIAAALVQIEPGAMRELHWHPNSDEWQYYLTGQGRMTVF |
| | OxdD | IGNGTARTFDYRAGDVGYVPSNAGHYIQNTGTETLWFLEMFKSDRYADVSLNQWMALTPK |
| | | ELVQGNLKAGSVMLDSLRKKKVPVVKYPST |

TABLE 2
Exemplary Formyl-coenzyme A transferases

| SEQ | Formyl- | |
|---|---|---|
| ID | coenzyme A | |
| NO: | transferase | Amino acid sequence |
| 5 | Lactobacillus | MTEEENEYAPLKGIKVVDWTQVQSGPSCTQILAWLGAEVIKIERTNIGDPTRNELL |
| | acidophilus | DIQDSWSLYYLQLNANKKSLTLNIKAPEGKKIMYDLLKKADIFVENIKPGAAEKAG |
| | strain La-14 Frc | YGWETVHKLNPRLIMASLKGFNEGSRFANVKAFEPVAQAAGGAASATGWNKGEFNV |
| | | PTQSAAALGDSNSGMHLTIAILAALMQREHTGEGTYVYQSMQDAVLNLCRIKLRDQ |
| | | LMLDNLGALPHYAVYPNYKWGDAIPRAENTEGGQVIGWTYKAKGWETDPNAYVYIV |
| | | VQNSNKSWEAIANTMGHPEWITDERFQDWQHRQLNKEALYQCIESYTKNYDKFELT |
| | | KTLGEAGIPVGPVLDWHELENDPDLNSDGTIVTIDQGGNRGKFKTIGLPFTLANYK |
| | | PDYKRAPDLGENNKEILSSLGYDPDQIEKLTEEGVISKAKGPKNPRVQVIKGE |
| 6 | Lactobacillus | MTEPNYNALEVNNTHLDKDKPYPLSGILVVDFTHVLSGPTCTRMLADAGARVIHIE |
| | acidophilus | RKTGDDTRHMRPYISDGSSEYFRICNAGKESVALDLKDPKDHALAEKMIAKADVVV |
| | strain La-14 | ENFRPGVMKRLGFGPEEMVKKYPKLIFASISGFGQYGPWSKQAAYDTIVQAVSGLM |
| | WP_003549134 | DATGTPKGKPTRVGTSVSDVVAGIMGYSAIMTALVARDRTGKGTTVDVSMLDSTES |
| | | LMVQDLMLALGPHEVPHRIGNRHPDMYPFDTFDCKDQPIAICCGNDHLWSLLSHTL |
| | | GHDEWVNQPNFKINDLREKNWQKVKNTMQAVLKTKNAAEWDKILHEAGIPAGLVLN |
| | | VDKTRRLDQIIARGMVKTLPDGNEVLGSPMKYSTWNSYGLQKDAPKLNENGDKIRK |
| | | EFE |
| 7 | Bifidobacterium | MADKSTAPLAGIKVIDWTQVQSGPSCTQILAWLGAEVIKLEKVHGGDPTRNEMNDV |
| | animalis subsp. | DGSYSLYFLQLNANKKSITLDMKDPEGKKILTDLLKDADVFVENIGPGDVEKLGFG |
| | lactis | WDEVHKINPKLIMASLKGFNQGSRFEHVKAFEPVAQCAGGAASTTGWWEGDKNIPT |
| | Frc | QSGAALGDSNTGMHLTIAILTALLQRERTGEGVFVYQSMQNAVLNLCRIKLRDQLI |
| | | LDHLHQLSYYDCYPGYKFGKAIPRAANAEGGLVLGWCYRAKGWETDPNAYVYIVIQ |
| | | QSQKGFENFCNAMGFQDWLTDPKFSTPNARDEHKQEVYKRVEEYTMQYDKYTLIKE |
| | | LGAKGVPVGPVLDWNELENDPDLNEDGTLITIDQGDARGKFKTIGLPFTMSNYAPD |
| | | YQRAPKLGENNEEILKSLGYTDEQIADLATKGVIGSNDGVKADLTAAPAQA |
| 8 | Oxalobacter | MTKPLDGINVLDFTHVQAGPACTQMMGFLGANVIKIERRGSGDMTRGWLQDKPNVD |
| | formigenes | SLYFTMFNCNKRSIELDMKTPEGKELLEQMIKKADVMVENFGPGALDRMGFTWEYI |
| | Frc | QELNPRVILASVKGYAEGHANEHLKVYENVAQCSGGAAATTGFWDGPPTVSGAALG |
| | | DSNSGMHLMIGILAALEMRHKTGRGQKVAVAMQDAVLNLVRIKLRDQQRLERTGIL |
| | | AEYPQAQPNFAFDRDGNPLSFDNITSVPRGGNAGGGGQPGWMLKCKGWETDADSYV |
| | | YFTIAANMWPQICDMIDKPEWKDDPAYNTFEGRVDKLMDIFSFIETKFADKDKFEV |
| | | TEWAAQYGIPCGPVMSMKELAHDPSLQKVGTVVEVVDEIRGNHLTVGAPFKFSGFQ |
| | | PEITRAPLLGEHTDEVLKELGLDDAKIKELHAKQVV |

TABLE 3
Exemplary Oxalyl-CoA decarboxylases

| SEQ | | |
|---|---|---|
| ID | Oxalyl-CoA | |
| NO: | decarboxylases | Amino acid sequence |
| 9 | Lactobacillus | MNLKCKMKAFLGFLKEGFFVVDTSLIGAALLIDALQANGLNNMYGVVGIPVTDFAR |
| | acidophilus | LAQLKGMKYYGFRREDSAVDAAAGAGFITGKPGVALTVSAPGFLNGLTALAQATKN |
| | strain La-14 | CFPLIMISGSSDRHIIDLDRGDYEGLDQYNVAKPFCKAAYRVDRAEDMGLAVARAV |
| | Oxc | RTAVSGRPGGVYLDLPAATVTDTVAQKSDANIYKVVDPAPKOLPSDDAINRAVELL |
| | | KDAKHPVILLGKGSAYAQSEDEIRELVNKINIPFLPMSMAKGVVPDDSPASAASAR |
| | | SFTLGQADVVLLIGARLNWMLSNGESPLFSEDAKFIQVDIDATEFDSNRKIDAPLQ |
| | | GDIKSVMQKLNSAAINAGVKAPTDWINAIKTESEKNNTKFAKRISASEAKSTLGYY |
| | | SAIEPINDLMQKHPDTYLVSEGANTLDIGRDLIGMQKPRHRLDIGTWGVMGVGMGY |
| | | AIAAAIETGKPVIALEGDSAFGFDGMEMETICRYHLPVIVVIINNGGIYNGDVNVV |
| | | PDQPGPTVLDHNAHYGDISKAFGGDSYRVNNYEEMKDALEKAYESGNPTIIDAQIP |
| | | ESMGKESGHIGNLNPKLDLSSLEAKENK |
| 10 | Bifidobacterium | MVDVSVTATSSDQNLIDSPHYLAETLIKNGVKHMYGVVGIPVTDFARIAQGMGIRF |
| | animalis subsp. | IGMRHEEDAVNAAAAEGFLTGRPAVALTVSAPGFLNGLAPLLEATINGFPVIMIGG |
| | Lactis | SSTRHVVDMHEGEYEGLDQMNYAKQFCKESFRIDKIEDIPLAVARAMHIACSGRPG |
| | Oxc | GVYIDFPDDAVAQTLDKDVAESQLWVANQPAPAMPPAQSSVDEALKLLSEAKNPLM |
| | | LVGKGAALAQAEDELREFVEKTDMPFQPMSMAKGVIPDDDPHCTASCRGLALRTAD |
| | | VVLLVGARLNWMLNFGEGKEWNPNVKFIQIDIDPNEIENARSIACPVVGDIKSAMQ |
| | | MINAGLEKTPVKASAQWLDMLKADAEKNDAKFAARVNSNTVPMGHYDALGAIKKVY |
| | | DQHKDMILTNEGANTLDDCRNIIDIYQPRHRLDCGTWGVMGCAVGYSIGAAVATGK |
| | | PVLYVGGDSGFGFDGMEVEVACRYNLPITFVVLNNGGIYRGDFENLGDDGDPSPLT |
| | | LSYDAHYERMIEAFGGNGYYATTPAEVEQMVGEAVASGKPSLVHVQLADYAGKESG |
| | | HISNLNPKPVVGPLATSEMTANPYLKGAHM |
| 11 | Oxalobacter | MSNDDNVELTDGFHVLIDALKMNDIDTMYGVVGIPITNLARMWQDDGQRFYSFRHE |
| | formigenes | QHAGYAASIAGYIEGKPGVCLTVSAPGFLNGVTSLAHATTNCFPMILLSGSSEREI |
| | Oxc | VDLQQGDYEEMDQMNVARPHCKASFRINSIKDIPIGIARAVRTAVSGRPGGVYVDL |
| | | PAKLFGQTISVEEANKLLFKPIDPAPAQIPAEDAIARAADLIKNAKRPVIMLGKGA |
| | | AYAQCDDEIRALVEETGIPFLPMGMAKGLLPDNHPQSAAATRAFALAQCDVCVLIG |
| | | ARLNWLMQHGKGKTWGDELKKYVQIDIQANEMDSNQPIAAPVVGDIKSAVSLLRKA |
| | | LKGAPKADAEWTGALKAKVDGNKAKLAGKMTAETPSGMMNYSNSLGVVRDFMLANP |
| | | DISLVNEGANALDNTRMIVDMLKPRKRLDSGTWGVMGIGMGYCVAAAAVTGKPVIA |
| | | VEGDSAFGFSGMELETICRYNLPVTVIIMNNGGIYKGNEADPQPGVISCTRLTRGR |
| | | YDMMMEAFGGKGYVANTPAELKAALEEAVASGKPCLINAMIDPDAGVESGRIKSLN |
| | | VVSKVGKK |

TABLE 4
Exemplary Oxalate transporters

| SEQ | | |
|---|---|---|
| ID | Oxalate | |
| NO: | transporter | Amino acid sequence |
| 12 | Lactobacillus | MTGKFEGLTQAEADKRLKEDGLNEVPEPEYNFFKEFLSKLWNLSAWILEGALILEC |
| | acidophilus | ILGKWVQSLFVLLMLLFAAFNGASKKKQSRRVLDTISHQLTPTVAVKRDGNWIKID |
| | strain La-14 | SKQLVKGDLISLQRGDVLAADVELVDGSIACDESSITGESKPVKKNVGDAAYAGTT |
| | WP_015613377 | IVEGDGLAIVTATGKNSRSGKTINLINNSAAPGHLQQLLTKIIYYLCLLDGVLTLV |
| | | IIIASFFKGGNFDTFINMLPFLAMMFIASIPVAMPSTFALSNSFEATRLSKEGVLT |
| | | SDLTGIQDAANLNLLLLDKTGTITENKTAVTSWTDLSSLPDKEVLALAGSATDKRN |
| | | AGIIDTAIDEYLTENNIPIMTAEKFTPFTSDTGYSMSIIDGHNVKLGSFKQLSLID |
| | | KNANEKIEGINFKAGRSVAVLIDDKLAGVFILQDKVRKDSKAALADLKKRGVRPIM |
| | | LTGDNQRTAAAVAEEVGLNGQVISIHDFNENTDIDDLAGIADVLPEDKLNMVKFFQ |
| | | QKGYIVGMTGDGVNDSPALKQAEVGIAVSNAADVAKRSGKMVLLDDGLGSIVKILD |
| | | AGHRVYQRMTTWSLIKLARTAELTMLLTFGYLFFNYIPMALNAMVIYTIMNNMVTM |
| | | MIGTDRTHITYKPENWNMAKLAKIAFSLAAGWTIIGFIFIWYLNTHGWSHGTISTM |
| | | VYVYLVLSAMLIVLITRTRKYFWQDYPSKMVGIVQIADVALTFILALCGLAMVQIS |
| | | WQNLLITIIVAVIAAILIDLVYQPVMKNR |
| 13 | Bifidobacterium | MTKILIDDIIPIIVIMALGYVCGKLSYFDNDQRQGLNKLVLNIALPAALFISIVKA |
| | animalis subsp. | TREMLAQDAVLTILGFIGIIVMFMLSYYLCRLMFHHSIQEAAVCALIAGSPTIGEL |
| | Lactis | GFAVLDPIYGDTVSTNLVIAIISIVVNAVTIPIGMYLINLGQSKDRERLSKAAVTN |
| | WP_004219149 | SKGQVSIANPKDDIAVDPNKDAKTDKTAEVMISKSSNMGKKKNQNLEALINALKQP |
| | | VCWAPLLAIVLVLIGVRVPSGFAPTFDLIAKANSGVAVLAAGLALSTVKFSLGWET |
| | | IWNTFYRLILTPAAFLGVGLLLGMGSNVNKLSMLVMAVALPPAFSGIIISSRYNIY |
| | | VKEGASTTAVSTVAFAVICLLWIWLVPLCCH |
| 14 | Oxalobacter | MNNPQTGQSTGLLGNRWFYLVLAVLLMCMISGVQYSWTLYANPVKDNLGVSLAAVQ |
| | formigenes | TAFTLSQVIQAGSQPGGGYFVDKFGPRIPLMFGGAMVLAGWTFMGMVDSVPALYAL |
| | oxalate | YTLAGAGVGIVYGIAMNTANRWFPDKRGLASGFTAAGYGLGVLPFLPLISSVLKVE |
| | formate | GVGAAFMYTGLIMGILIILIAFVIRFPGQQGAKKQIVVTDKDENSGEMLRTPQFWV |
| | antiporter | LWTAFFSVNFGGLLLVANSVPYGRSLGLAAGVLTIGVSIQNLENGGCRPFWGFVSD |
| | | KIGRYKTMSVVFGINAVVLALFPTIAALGDVAFIAMLAIAFFTWGGSYALFPSTNS |
| | | DIFGTAYSARNYGFFWAAKATASIFGGGLGAAIATNFGWNTAFLITAITSFIAFAL |
| | | ATFVIPRMGRPVKKMVKLSPEEKAVH |

TABLE 5
Exemplary integrative shuttle vector sequences

| SEQ | | |
|---|---|---|
| ID | | |
| NO: | | Nucleotide sequence |
| 15 | Bacillus subtilis | tacatgctggctcaaatcatcctgcctgtctaaaaattcttcaaatgcatcgctacctgtttcggaagaaagccgatctcccgcctccggat |
| | PY79 | gatcaaccgtcagtggataccgtttttggtttttgcgaacccgcttatcaaaatcaatagaaaagatgcgaatcattgaattcatatacttgac |
| | NBP3510: | gatgcggatttctttataaaattgcttaaatagctcgtccaatgatcttgcatctttttcgttagggctttccattacgtttttgaacaaacggtaat |
| | oxdC | gctgaggattgctcaaaaaatgcttcacgatgggatgcttcattatctccttacattttgaccgtaaaagactttgatagtccatcaaaccgcc |
| | Integrative | tccctactaaattaaatgaaggaaagcaaaaaaaagtaaactactactttccgcctagtgtcttccaaaatactttgcagcagtttacgttttat |
| | vector fragment | cggttttcctcacttacatacatgactgcagaaaaagaagggaggtattttcctatggatcaggtttttatagaggaagtcgtaaaacagatc |
| | | ggcaatttggggtttcccgcgctgattgcaatgtatctgctgacccgattcgaaaagaagtttgatcaactaatagaactaatgacagaact |
| | | gaaagatcatgcaaaaaaataatttttcaatcgaagttgacttttcactggtttttttcacttaacaaaacagaagggaaaacgaaaggccttt |
| | | caccttctctttctgctatcacatttaaatgtaaggaggaaacatttcacttctcaaagatcccatttaaaaattttttttaaaaaaatatttgacat |
| | | ttttaaataaagcgtttataatatatgtagaaacaacaaagggggagatttgtttgataaggaggacaaacatgaaaaaacaaaatgacatt |
| | | ccgcagccaattagaggagacaaaggagcaacggtaaaaatcccgcgcaatattgaaagagaccggcaaaaccctgatatgctcgtt |
| | | ccgcctgaaaccgatcatggcaccgtcagcaatatgaagttttcattctctgatactcataaccgattagaaaaaggcggatatgcccggg |
| | | aagtgacagtacgtgaattgccgatttcagaaaaccttgcatccgtaaatatgcggctgaagccaggcgcgattcgcgagcttcactggc |
| | | ataaagaagctgaatgggcttatatgatttacggaagtgcaagagtcacaattgtagatgaaaaagggcgcagctttattgacgatgtagg |
| | | tgaaggagacctttggtacttcccgtcaggcctgccgcactccatccaagcgctggaggagggagctgagttcctgctcgtgtttgacga |
| | | tggatcattctctgaaaacagcacgttccagctgacagattggctggcccacactccaaaagaagtcattgctgcgaacttcggcgtgac |
| | | aaaagaagagatttccaatttgcctggcaaagaaaaatatatatttgaaaaccaacttcctggcagtttaaaagatgatattgtggaagggc |
| | | cgaatggcgaagtgccttatccatttacttaccgccttcttgaacaagagccgatcgaatctgagggaggaaaagtatacattgcagattc |
| | | gacaaacttcaaagtgtctaaaaccatcgcatcagcgctcgtaacagtagaaccc |
| 16 | Bacillus subtilis | cttgactcaatgaaacaaaaaatgcatttaagagctgttatacgttttaatcttgtctttagaaagcactttaatttaagtcaaaagtttagacag |
| | PY79 | tcatctgttagatcattagccctttcttattttcttttcggtaacattttgatcgtattttcctactaaaaagttaggaaagtgaggaaaaacatga |
| | NBP3510: | gaaaaaagaacaatattaagaaatggctattgatcattgctggattcttgatcatctgcatcatcacattatttgtaatggtgtcagggaacaa |
| | oxdD | agtgaaatatgagggcagcgggaaaagcggactgtggatgtctaaccttgaaaaatcagacaaaacttcaattggacccaattattttcta |
| | Integrative | aatctatattggcaggggagtaagaaggaagaaaagtggactgttgttgaaagaattactctgtatgtagatggcgaaaagtatcaagat |
| | vector fragment | gataacgtagatgagtacgacttatcggagtacacaggtgatgaaatgccggggggggacgcatggaggaccatatttccacctttgat |
| | | tatatgcctgaagatgaagtgataggtcatgatgtattagtaaaagtggagtggaggacaggccagaaaaaacagacagaagcaatca |
| | | aattacataagaagccatggtataaaaaatagtttatttgatgtatttgtgatcacattggtggtcacttttttatttgcggattcctaggcacag |
| | | caatctaagattctgcataggctgaaataaaatcttgttcatttctaaaacgaggtgcacttctcaaagatcccatttaaaaattttttttaaaaa |
| | | aatatttgacatttttaaataaagcgtttataatatatgtagaaacaacaaagggggagatttgtttgataaggaggacaaacatgctgttgg |
| | | aacaacaaccaatcaatcatgaagacagaaacgtgccgcagcctattcgaagtgatggagctggagctattgatacaggcccgcgaaa |
| | | tataatacgggatattcaaaatccgaatatatttgttccgcctgttacagatgagggtatgattcctaacttgagattttcattctcagacgctc |
| | | ccatgaaattagatcacggcggctggtcaagagaaatcaccgtaagacagcttccgatttcgactgcgattgcaggtgtaaacatgagct |
| | | taactgcgggaggcgtccgcgagcttcattggcataagcaagcggagtgggcttatatgcttttgggacgggcacgtatcaccgctgttg |
| | | accaagacggacgaaatttcattgctgatgttggtcccggcgacctttggtacttcccggcaggaattccgcattccatacagggattgga |
| | | acactgcgagtttctgctcgttttcgatgatgggaacttttctgagttttcaacgttaaccatttcagattggcttgcacacacaccaaaagat |
| | | gttctgtctgcaaatttcggtgtcccggagaatgctttcaactctcttccgtctgagcaagtctatatctaccaagggaatgtgccgggatca |
| | | gtcgccagtgaagacattcagtcaccatatggaaaagtccccatgacctttaaacacgagctgttaaatcaacccccaattcaaatgcca |
| | | ggggggagtgtacgaattgtggattcttctaacttcccaatttcaaaaacgatagccgc |
| 17 | Bacillus subtilis | aaatgcttcacgatgggatgcttcattatctccttacattttgaccgcaaaagaccttgatagtccatcaaaccgcctccctactaaattaaat |
| | subsp. | gaaggaaagcaaaaaaaagtaaactactactttctgactagcgcttgtgcaaatgctccggctgcagtttacgttttcttggatttccttcact |
| | inaquosorum | tacatacatgactgccaaaaaagaagggaggtattttccatggatcaggtttttatagaggaagtcgtaaaacagatcggtaatctggggtt |
| | strain | tcctgcgctgattgcaatgtacctgctgacccgatttgagaaaaagtttgatcagctgatagaactgatgacagagctgaaagaccagaa |
| | DE111 ® | agcaaaaaattaattttcaatcgaagttgactttttactgttttttttcacttaacaagacagaagggaaaacgaaaggcctttcaccttctcttt |
| | NBP3510: | ctgcttaacatttatttttaaggaggagacagttacacttctcaaagatcccatttaaaaattttttttaaaaaaatatttgacatttttaaataaag |
| | oxdC | cgtttataatatatgtagaaacaacaaagggggagatttgtttgataaggaggacaaacatgaaaaaacaaaacgacattccgcagccaa |
| | Integrative | tcagaggagacaaaggagccacggtgaaaatcccgcgcaatattgaaagagaccggcaaaatcctgatatgctcgttccgccggaaa |
| | vector fragment | ccgatcatggaaccgtcagcaatatgaaattttcgttctctgatactcataaccggttagaaaaaggcggatatgcccgtgaagtcaccgt |
| | | acgggagctgccgatttcagagaaccttgcatctgtaaatatgcggctgaagccaggtgccattcgcgagctgcattggcataaagaag |
| | | cagaatgggcttatatgatttacggaagtgctagagtcacaattgtggatgaaaaagggcgcagctttattgatgatgtaggtgaaggaga |
| | | tctttggtacttcccgtcaggcctgccgcactccattcaagcgctggatgaaggagccgagttcctgctcgtgtttgacgatggatcattct |
| | | ctgaaaacagcacgttccagctgacagactggcttgcccacacccctaaagaagtcattgctgcgaactttggcgtgacaaaagaagaa |
| | | atcgccaatctgccgggtaaagaaaaatatatatttgaaaaccaaatcccaggcagcttaaaagatgacattgtggaagggccgaacgg |
| | | cgaagtgccttacccgtttacttaccgccttcttgaacaggagccgattgaatcagagggaggaaaagtatacattgcggattcgacaaa |
| | | ctttacagtgtctaaaacaatcgcatctgccctcgtgacggtagaactaaatagatag |
| 18 | Bacillus subtilis | gccgctatgttttttgaaatcgggaagttagaagagtccacaattcgtacgctccccccaggcgtttgaatgggggtttgatttaataactcg |
| | subsp. | tatttgaaagtcaaggggacttttccgtaaggtgactgaatgtcttcactggcgactgatcccggcacatttccttgataaatatagacttgtt |
| | inaquosorum | ctgaaggaagagagtgaaaagcattctctgggacaccaaaatttccagacagaacatcttttggcgtgtgtgcaagccaatctgaaatgg |
| | strain | ttaatgttgaaaactcagaaaagtttccatcatcgaagacaagcagaaattcgcaatgttctaatccttgaatggaatgcggaattcctgcg |
| | DE111 ® | ggaaagtaccaaagatcgcctggaccaacatcagcaatgaaatttcgcccctcttggtcaactgcggtgatgcgggcccgtcccaaaa |
| | NBP3510: | gcatataagcccattccgcttgtttatgccaatgaagttcacgtacgcctcccgcagttaagctcatatttactcctgcaattgcggtcgaaat |
| | oxdD | cggaagctgcctgacggtgatttcacgtgaccagccgccgtgatctaatttcatcggagcatctgagaatgaaaatctcaagttcggaatc |
| | Integrative | ataccttcgtctgtaacaggcggaacaagtatatttggattttgaatatctcgcatcatattacgcgggccagcatcaatagccccggctcc |
| | vector fragment | atcacttcgaataggctgcggcacgtttctgtcttcatgattgattggttgtttttccaacagcatgtttgtcctccttatcaaacaaatctcccc |
| | | ctttgttgtttctacatatattataaacgctttatttaaaaatgtcaaatatttttttaaaaaaaatttttaaatgggatctttgagaagtgcacctcgt |
| | | tttagaaattaacaagattttattccagcctatgcatgatcttatatggctgtgcctagaattcagcaaataagctatcttttataccatggattct |
| | | tatgtaatttgatttcttctgtctgttctttccggcctgtcttccactctactttcactaagacatcatgtcctatcacttcatcttcaggcatataatt |
| | | aaaggtagcgatatggtcctccatgtgtcctccaccttgcatttcttcaccagtgtattctgataagtcgtactcatctatgttatcatcttgata |
| | | ctttttgccatctacatacagagtaattctttcaacaacagtccttttctcgtctttcttacttccttgccaatacaaatttagaaagtaattaggtc |
| | | caattgacgtcttgtctgatttttcaaggttagaaacccacagtccgcttcttccgctgccttcatatttcactttgtttcctgagatcattacaat |
| | | gattgctacgatgcagattattaagaatgcagcaatcatcaatagccattttttaatgttgttcttttttctcatgattttcctcacttccctcatact |
| | | tagtaggaaaatggggtcaaaatgttactgataagaacttaaattaaagcgttataggggcaagattaaaacttataaacatgctcttacatg |
| | | cattttttatttcattgagtcaattaccttcaaaatgattacattctttaacaaaatcccctatgattgtggtaatatctttgtgagatatttttgaattt |
| | | tgagaaagag |

1. An engineered microbial cell, the microbial cell comprising: an exogenous nucleic acid encoding at least one oxalate catabolismenzyme.

2. The engineered microbial cell of claim 1, wherein the engineered microbial cell is a bacterial cell.

3. The engineered microbial cell of claim 1, wherein the encoded oxalate catabolismenzyme is constitutively expressed in the engineered microbial cell.

4. The engineered microbial cell of claim 1, wherein the at least one oxalate catabolismenzyme is an oxalate decarboxylase that directly catalyzes the reaction: oalate+H⁺ formate+CO₂.

5. The engineered microbial cell of claim 1, wherein the at least one oxalate catabolismenzyme is a bacterial oxalate decarboxylase.

6. The engineered microbial cell of claim 1, wherein the at least one oxalate catabolismenzyme comprises at least 95% identity with the full length of any one of SEQ ID NOs: 1-4.

7. The engineered microbial cell of claim 6, wherein the oxalate decarboxylase is a mutant oxalate decarboxylase, a chimera, a and/or an overexpression variant of the oxalate decarboxylase.

8. The engineered microbial cell of claim 1, wherein the engineered microbial cell is present in a composition comprising an enteric coating and/or a preservative.

9. The engineered microbial cell of claim 8, wherein the composition is a pharmaceutical composition.

10. The engineered microbial cell of claim 9, wherein the pharmaceutical composition comprises a pharmaceutically acceptable carrier.

11. The engineered microbial cell of claim 8, wherein the engineered microbial cell is located within the enteric coating.

12. A method of administering a microbial cell to a subject, the method comprising administering to the gastrointestinal tract of the subject the engineered microbial cell of claim 1.

13. The method according to claim 12, wherein the administration of the engineered microbial cell lowers the level of oxalate in the gastrointestinal tract as compared to the level of level of oxalate in the gastrointestinal tract prior to administration.

14. The method according to claim 12, wherein the administration is oral administration.

15. The method according to claim 12, wherein the subject suffers from a condition selected from the group consisting of: hyperoxaluria, hyperoxalemia, calcium oxalate nephrocalcinosis, calcium oxalate nephrolithiasis, calcium oxalate urolithiasis, and combinations thereof.

16. The method according to claim 12, wherein administering the engineered microbial cell comprises administering 10\hat{ }6 to 10\hat{ }13 of the engineered microbial cells.

17. The method according to claim 12, wherein administering the engineered microbial cell of claim 1 comprises administering about 10\hat{ }9 of the engineered microbial cells

18. A method for selecting cells expressing extracellular oxalate decarboxylase the method comprising:
Suspending in an assay buffer comprising oxalate a known known number of microbial cells;
incubating the microbial cells in the assay buffer under conditions suitable for oxalate degradation;
measuring the formate concentration in the assay buffer, and
selecting microbial cells producing higher levels of formate.


SEQUENCE LISTING (18 sequences)

SEQ ID NO: 1 - protein, 385 aa, Bacillus subtilis
MKKQNDIPQPIRGDKGATVKIPRNIERDRQNPDMLVPPETDHGTVSNMKFSFSDTHNRLEKGGYAREVTVRELPISENLASVNMRLKPGAIRELHWHKEAEWAYMIYGSARVTIVDEKGRSFIDDVGEGDLWYFPSGLPHSIQALEEGAEFLLVFDDGSFSENSTFQLTDWLAHTPKEVIAANFGVTKEEISNLPGKEKYIFENQLPGSLKDDIVEGPNGEVPYPFTYRLLEQEPIESEGGKVYIADSTNFKVSKTIASALVTVEPGAMRELHWHPNTHEWQYYISGKARMTVFASDGHARTFNYQAGDVGYVPFAMGHYVENIGDEPLVFLEIFKDDHYADVSLNQWLAMLPETFVQAHLDLGKDFTDVLSKEKHPVVKKKCSK

SEQ ID NO: 2 - protein, 392 aa, Bacillus subtilis
MLLEQQPINHEDRNVPQPIRSDGAGAIDTGPRNIIRDIQNPNIFVPPVTDEGMIPNLRFSFSDAPMKLDHGGWSREITVRQLPISTAIAGVNMSLTAGGVRELHWHKQAEWAYMLLGRARITAVDQDGRNFIADVGPGDLWYFPAGIPHSIQGLEHCEFLLVFDDGNFSEFSTLTISDWLAHTPKDVLSANFGVPENAFNSLPSEQVYIYQGNVPGSVASEDIQSPYGKVPMTFKHELLNQPPIQMPGGSVRIVDSSNFPISKTIAAALVQIEPGAMRELHWHPNSDEWQYYLTGQGRMTVFIGNGTARTFDYRAGDVGYVPSNAGHYIQNTGTETLWFLEMFKSNRYADVSLNQWMALTPKELVQSNLNAGSVMLDSLRKKKVPVVKYPGT

SEQ ID NO: 3 - protein, 385 aa, Bacillus subtilis
MKKQNDIPQPIRGDKGATVKIPRNIERDRQNPDMLVPPETDHGTVSNMKFSFSDTHNRLEKGGYAREVTVRELPISENLASVNMRLKPGAIRELHWHKEAEWAYMIYGSARVTIVDEKGRSFIDDVGEGDLWYFPSGLPHSIQALDEGAEFLLVFDDGSFSENSTFQLTDWLAHTPKEVIAANFGVTKEEIANLPGKEKYIFENQIPGSLKDDIVEGPNGEVPYPFTYRLLEQEPIESEGGKVYIADSTNFTVSKTIASALVTVEPGAMRELHWHPNTHEWQYYISGKARMTVFASDGHARTFNYQAGDVGYVPFAMGHYVENIGDEPLVFLEIFKDDHYADVSLNQWLAMLPEKFVQAHLDLGKDFTDVLSKEKHPVVKKKCSK

SEQ ID NO: 4 - protein, 390 aa, Bacillus subtilis
MEKQPINHEDRNVPQPIRSDGAGAIDAGPRNMMRDIQNPNILVPPVTDEGMIPNLRFSFSDAPMKLDHGGWSREITVRQLPISTAIAGVNMSLTAGGVRELHWHKQAEWAYMLLGRARITAVDQEGRNFIADVGPGDLWYFPAGIPHSIQGLEHCEFLLVFDDGNFSEFSTLTISDWLAHTPKDVLSGNFGVPENAFHSLPSEQVYIYQGNVPGSVASEDIQSPYGKVPLTFKYELLNQTPIQTPGGSVRIVDSSNFPISKNIAAALVQIEPGAMRELHWHPNSDEWQYYLTGQGRMTVFIGNGTARTFDYRAGDVGYVPSNAGHYIQNTGTETLWFLEMFKSDRYADVSLNQWMALTPKELVQGNLKAGSVMLDSLRKKKVPVVKYPST

SEQ ID NO: 5 - protein, 445 aa, Lactobacillus acidophilus
MTEEENEYAPLKGIKVVDWTQVQSGPSCTQILAWLGAEVIKIERTNTGDPTRNELLDIQDSWSLYYLQLNANKKSLTLNIKAPEGKKIMYDLLKKADIFVENIKPGAAEKAGYGWETVHKLNPRLIMASLKGFNEGSRFANVKAFEPVAQAAGGAASATGWNKGEFNVPTQSAAALGDSNSGMHLTIAILAALMQREHTGEGTYVYQSMQDAVLNLCRIKLRDQLMLDNLGALPHYAVYPNYKWGDAIPRAENTEGGQVIGWTYKAKGWETDPNAYVYIVVQNSNKSWEAIANTMGHPEWITDERFQDWQHRQLNKEALYQCIESYTKNYDKFELTKTLGEAGIPVGPVLDWHELENDPDLNSDGTIVTIDQGGNRGKFKTIGLPFTLANYKPDYKRAPDLGENNKEILSSLGYDPDQIEKLTEEGVISKAKGPKNPRVQVIKGE

SEQ ID NO: 6 - protein, 395 aa, Lactobacillus acidophilus
MTEPNYNALEVNNTHLDKDKPYPLSGILVVDFTHVLSGPTCTRMLADAGARVIHIERKTGDDTRHMRPYISDGSSEYFRICNAGKESVALDLKDPKDHALAEKMIAKADVVVENFRPGVMKRLGFGPEEMVKKYPKLIFASISGFGQYGPWSKQAAYDTIVQAVSGLMDATGTPKGKPTRVGTSVSDVVAGIMGYSAIMTALVARDRTGKGTTVDVSMLDSTFSLMVQDLMLALGPHEVPHRIGNRHPDMYPFDTFDCKDQPIAICCGNDHLWSLLSHTLGHDEWVNQPNFKTNDLREKNWQKVKNTMQAVLKTKNAAEWDKILHEAGIPAGLVLNVDKTRRLDQIIARGMVKTLPDGNEVLGSPMKYSTWNSYGLQKDAPKLNENGDKIRKEFE

SEQ ID NO: 7 - protein, 443 aa, Bifidobacterium animalis
MADKSTAPLAGIKVIDWTQVQSGPSCTQILAWLGAEVIKLEKVHGGDPTRNEMNDVDGSYSLYFLQLNANKKSITLDMKDPEGKKILTDLLKDADVFVENIGPGDVEKLGFGWDEVHKINPKLIMASLKGFNQGSRFEHVKAFEPVAQCAGGAASTTGWWEGDKNIPTQSGAALGDSNTGMHLTIAILTALLQRERTGEGVFVYQSMQNAVLNLCRIKLRDQLILDHLHQLSYYDCYPGYKFGKAIPRAANAEGGLVLGWCYRAKGWETDPNAYVYIVIQQSQKGFENFCNAMGFQDWLTDPKFSTPNARDEHKQEVYKRVEEYTMQYDKYTLTKELGAKGVPVGPVLDWNELENDPDLNEDGTLITIDQGDARGKFKTIGLPFTMSNYAPDYQRAPKLGENNEEILKSLGYTDEQIADLATKGVIGSNDGVKADLTAAPAQA

SEQ ID NO: 8 - protein, 428 aa, Oxalobacter formigenes
MTKPLDGINVLDFTHVQAGPACTQMMGFLGANVIKIERRGSGDMTRGWLQDKPNVDSLYFTMFNCNKRSIELDMKTPEGKELLEQMIKKADVMVENFGPGALDRMGFTWEYIQELNPRVILASVKGYAEGHANEHLKVYENVAQCSGGAAATTGFWDGPPTVSGAALGDSNSGMHLMIGILAALEMRHKTGRGQKVAVAMQDAVLNLVRIKLRDQQRLERTGILAEYPQAQPNFAFDRDGNPLSFDNITSVPRGGNAGGGGQPGWMLKCKGWETDADSYVYFTIAANMWPQICDMIDKPEWKDDPAYNTFEGRVDKLMDIFSFIETKFADKDKFEVTEWAAQYGIPCGPVMSMKELAHDPSLQKVGTVVEVVDEIRGNHLTVGAPFKFSGFQPEITRAPLLGEHTDEVLKELGLDDAKIKELHAKQVV

SEQ ID NO: 9 - protein, 588 aa, Lactobacillus acidophilus
MNLKCKMKAFLGFLKEGFFVVDTSLTGAALLIDALQANGLNNMYGVVGIPVTDFARLAQLKGMKYYGFRREDSAVDAAAGAGFITGKPGVALTVSAPGFLNGLTALAQATKNCFPLIMISGSSDRHIIDLDRGDYEGLDQYNVAKPFCKAAYRVDRAEDMGLAVARAVRTAVSGRPGGVYLDLPAATVTDTVAQKSDANIYKVVDPAPKQLPSDDAINRAVELLKDAKHPVILLGKGSAYAQSEDEIRELVNKTNIPFLPMSMAKGVVPDDSPASAASARSFTLGQADVVLLIGARLNWMLSNGESPLFSEDAKFIQVDIDATEFDSNRKIDAPLQGDIKSVMQKLNSAAINAGVKAPTDWINAIKTESEKNNTKFAKRISASEAKSTLGYYSAIEPINDLMQKHPDTYLVSEGANTLDIGRDLIGMQKPRHRLDTGTWGVMGVGMGYAIAAAIETGKPVIALEGDSAFGFDGMEMETICRYHLPVIVVIINNGGIYNGDVNVVPDQPGPTVLDHNAHYGDISKAFGGDSYRVNNYEEMKDALEKAYESGNPTIIDAQIPESMGKESGHIGNLNPKLDLSSLEAKENK

SEQ ID NO: 10 - protein, 590 aa, Bifidobacterium animalis
MVDVSVTATSSDQNLTDSPHYLAETLIKNGVKHMYGVVGIPVTDFARIAQGMGIRFIGMRHEEDAVNAAAAEGFLTGRPAVALTVSAPGFLNGLAPLLEATTNGFPVIMIGGSSTRHVVDMHEGEYEGLDQMNYAKQFCKESFRIDKIEDIPLAVARAMHIACSGRPGGVYIDFPDDAVAQTLDKDVAESQLWVANQPAPAMPPAQSSVDEALKLLSEAKNPLMLVGKGAALAQAEDELREFVEKTDMPFQPMSMAKGVIPDDDPHCTASCRGLALRTADVVLLVGARLNWMLNFGEGKEWNPNVKFIQIDIDPNEIENARSIACPVVGDIKSAMQMINAGLEKTPVKASAQWLDMLKADAEKNDAKFAARVNSNTVPMGHYDALGAIKKVYDQHKDMILTNEGANTLDDCRNIIDIYQPRHRLDCGTWGVMGCAVGYSIGAAVATGKPVLYVGGDSGFGFDGMEVEVACRYNLPITFVVLNNGGIYRGDFENLGDDGDPSPLTLSYDAHYERMIEAFGGNGYYATTPAEVEQMVGEAVASGKPSLVHVQLADYAGKESGHISNLNPKPVVGPLATSEMTANPYLKGAHM

SEQ ID NO: 11 - protein, 512 aa, Oxalobacter formigenes
QHAGYAASIAGYIEGKPGVCLTVSAPGFLNGVTSLAHATTNCFPMILLSGSSEREIVDLQQGDYEEMDQMNVARPHCKASFRINSIKDIPIGIARAVRTAVSGRPGGVYVDLPAKLFGQTISVEEANKLLFKPIDPAPAQIPAEDAIARAADLIKNAKRPVIMLGKGAAYAQCDDEIRALVEETGIPFLPMGMAKGLLPDNHPQSAAATRAFALAQCDVCVLIGARLNWLMQHGKGKTWGDELKKYVQIDIQANEMDSNQPIAAPVVGDIKSAVSLLRKALKGAPKADAEWTGALKAKVDGNKAKLAGKMTAETPSGMMNYSNSLGVVRDFMLANPDISLVNEGANALDNTRMIVDMLKPRKRLDSGTWGVMGIGMGYCVAAAAVTGKPVIAVEGDSAFGFSGMELETICRYNLPVTVIIMNNGGIYKGNEADPQPGVISCTRLTRGRYDMMMEAFGGKGYVANTPAELKAALEEAVASGKPCLINAMIDPDAGVESGRIKSLNVVSKVGKK

SEQ ID NO: 12 - protein, 757 aa, Lactobacillus acidophilus
MTGKFEGLTQAEADKRLKEDGLNEVPEPEYNFFKEFLSKLWNLSAWILEGALILECILGKWVQSLFVLLMLLFAAFNGASKKKQSRRVLDTISHQLTPTVAVKRDGNWIKIDSKQLVKGDLISLQRGDVLAADVELVDGSIACDESSITGESKPVKKNVGDAAYAGTTIVEGDGLAIVTATGKNSRSGKTINLINNSAAPGHLQQLLTKIIYYLCLLDGVLTLVIIIASFFKGGNFDTFINMLPFLAMMFIASIPVAMPSTFALSNSFEATRLSKEGVLTSDLTGIQDAANLNLLLLDKTGTITENKTAVTSWTDLSSLPDKEVLALAGSATDKRNAGIIDTAIDEYLTENNIPIMTAEKFTPFTSDTGYSMSIIDGHNVKLGSFKQLSLIDKNANEKIEGINFKAGRSVAVLIDDKLAGVFILQDKVRKDSKAALADLKKRGVRPIMLTGDNQRTAAAVAEEVGLNGQVISIHDFNENTDIDDLAGIADVLPEDKLNMVKFFQQKGYIVGMTGDGVNDSPALKQAEVGIAVSNAADVAKRSGKMVLLDDGLGSIVKILDAGHRVYQRMTTWSLTKLARTAELTMLLTFGYLFFNYIPMALNAMVIYTIMNNMVTMMIGTDRTHITYKPENWNMAKLAKIAFSLAAGWTIIGFIFIWYLNTHGWSHGTISTMVYVYLVLSAMLIVLITRTRKYFWQDYPSKMVGIVQIADVALTFILALCGLAMVQISWQNLLITIIVAVIAAILIDLVYQPVMKNR

SEQ ID NO: 13 - protein, 367 aa, Bifidobacterium animalis
MTKILIDDIIPIIVIMALGYVCGKLSYFDNDQRQGLNKLVLNIALPAALFISIVKATREMLAQDAVLTILGFIGIIVMFMLSYYLCRLMFHHSIQEAAVCALIAGSPTIGFLGFAVLDPIYGDTVSTNLVIAIISIVVNAVTIPIGMYLINLGQSKDRERLSKAAVTNSKGQVSIANPKDDIAVDPNKDAKTDKTAEVMISKSSNMGKKKNQNLEALINALKQPVCWAPLLAIVLVLIGVRVPSGFAPTFDLIAKANSGVAVLAAGLALSTVKFSLGWETIWNTFYRLILTPAAFLGVGLLLGMGSNVNKLSMLVMAVALPPAFSGIIISSRYNIYVKEGASTTAVSTVAFAVTCLLWIWLVPLCCH

SEQ ID NO: 14 - protein, 418 aa, Oxalobacter formigenes
MNNPQTGQSTGLLGNRWFYLVLAVLLMCMISGVQYSWTLYANPVKDNLGVSLAAVQTAFTLSQVIQAGSQPGGGYFVDKFGPRIPLMFGGAMVLAGWTFMGMVDSVPALYALYTLAGAGVGIVYGIAMNTANRWFPDKRGLASGFTAAGYGLGVLPFLPLISSVLKVEGVGAAFMYTGLIMGILIILIAFVIRFPGQQGAKKQIVVTDKDFNSGEMLRTPQFWVLWTAFFSVNFGGLLLVANSVPYGRSLGLAAGVLTIGVSIQNLFNGGCRPFWGFVSDKIGRYKTMSVVFGINAVVLALFPTIAALGDVAFIAMLAIAFFTWGGSYALFPSTNSDIFGTAYSARNYGFFWAAKATASIFGGGLGAAIATNFGWNTAFLITAITSFIAFALATFVIPRMGRPVKKMVKLSPEEKAVH

SEQ ID NO: 15 - DNA, 1719 bp, Bacillus subtilis
  source 1..1719: mol_type = genomic DNA
tacatgctggctcaaatcatcctgcctgtctaaaaattcttcaaatgcatcgctacctgtttcggaagaaagccgatctcccgcctccggatgatcaaccgtcagtggataccgtttttggtttttgcgaacccgcttatcaaaatcaatagaaaagatgcgaatcattgaattcatatacttgacgatgcggatttctttataaaattgcttaaatagctcgtccaatgatcttgcatctttttcgttagggctttccattacgtttttgaacaaacggtaatgctgaggattgctcaaaaaatgcttcacgatgggatgcttcattatctccttacattttgaccgtaaaagactttgatagtccatcaaaccgcctccctactaaattaaatgaaggaaagcaaaaaaaagtaaactactactttccgcctagtgtcttccaaaatactttgcagcagtttacgttttatcggttttcctcacttacatacatgactgcagaaaaagaagggaggtattttcctatggatcaggtttttatagaggaagtcgtaaaacagatcggcaatttggggtttcccgcgctgattgcaatgtatctgctgacccgattcgaaaagaagtttgatcaactaatagaactaatgacagaactgaaagatcatgcaaaaaaataatttttcaatcgaagttgacttttcactggtttttttcacttaacaaaacagaagggaaaacgaaaggcctttcaccttctctttctgctatcacatttaaatgtaaggaggaaacatttcacttctcaaagatcccatttaaaaattttttttaaaaaaatatttgacatttttaaataaagcgtttataatatatgtagaaacaacaaagggggagatttgtttgataaggaggacaaacatgaaaaaacaaaatgacattccgcagccaattagaggagacaaaggagcaacggtaaaaatcccgcgcaatattgaaagagaccggcaaaaccctgatatgctcgttccgcctgaaaccgatcatggcaccgtcagcaatatgaagttttcattctctgatactcataaccgattagaaaaaggcggatatgcccgggaagtgacagtacgtgaattgccgatttcagaaaaccttgcatccgtaaatatgcggctgaagccaggcgcgattcgcgagcttcactggcataaagaagctgaatgggcttatatgatttacggaagtgcaagagtcacaattgtagatgaaaaagggcgcagctttattgacgatgtaggtgaaggagacctttggtacttcccgtcaggcctgccgcactccatccaagcgctggaggagggagctgagttcctgctcgtgtttgacgatggatcattctctgaaaacagcacgttccagctgacagattggctggcccacactccaaaagaagtcattgctgcgaacttcggcgtgacaaaagaagagatttccaatttgcctggcaaagaaaaatatatatttgaaaaccaacttcctggcagtttaaaagatgatattgtggaagggccgaatggcgaagtgccttatccatttacttaccgccttcttgaacaagagccgatcgaatctgagggaggaaaagtatacattgcagattcgacaaacttcaaagtgtctaaaaccatcgcatcagcgctcgtaacagtagaaccc

SEQ ID NO: 16 - DNA, 1721 bp, Bacillus subtilis
  source 1..1721: mol_type = genomic DNA
cttgactcaatgaaacaaaaaatgcatttaagagctgttatacgttttaatcttgtctttagaaagcactttaatttaagtcaaaagtttagacagtcatctgttagatcattagccctttcttattttcttttcggtaacattttgatcgtattttcctactaaaaagttaggaaagtgaggaaaaacatgagaaaaaagaacaatattaagaaatggctattgatcattgctggattcttgatcatctgcatcatcacattatttgtaatggtgtcagggaacaaagtgaaatatgagggcagcgggaaaagcggactgtggatgtctaaccttgaaaaatcagacaaaacttcaattggacccaattattttctaaatctatattggcaggggagtaagaaggaagaaaagtggactgttgttgaaagaattactctgtatgtagatggcgaaaagtatcaagatgataacgtagatgagtacgacttatcggagtacacaggtgatgaaatgccgggtgggggacgcatggaggaccatatttccacctttgattatatgcctgaagatgaagtgataggtcatgatgtattagtaaaagtggagtggaggacaggccagaaaaaacagacagaagcaatcaaattacataagaagccatggtataaaaaatagtttatttgatgtatttgtgatcacattggtggtcacttttttatttgcggattcctaggcacagcaatctaagattctgcataggctgaaataaaatcttgttcatttctaaaacgaggtgcacttctcaaagatcccatttaaaaattttttttaaaaaaatatttgacatttttaaataaagcgtttataatatatgtagaaacaacaaagggggagatttgtttgataaggaggacaaacatgctgttggaacaacaaccaatcaatcatgaagacagaaacgtgccgcagcctattcgaagtgatggagctggagctattgatacaggcccgcgaaatataatacgggatattcaaaatccgaatatatttgttccgcctgttacagatgagggtatgattcctaacttgagattttcattctcagacgctcccatgaaattagatcacggcggctggtcaagagaaatcaccgtaagacagcttccgatttcgactgcgattgcaggtgtaaacatgagcttaactgcgggaggcgtccgcgagcttcattggcataagcaagcggagtgggcttatatgcttttgggacgggcacgtatcaccgctgttgaccaagacggacgaaatttcattgctgatgttggtcccggcgacctttggtacttcccggcaggaattccgcattccatacagggattggaacactgcgagtttctgctcgttttcgatgatgggaacttttctgagttttcaacgttaaccatttcagattggcttgcacacacaccaaaagatgttctgtctgcaaatttcggtgtcccggagaatgctttcaactctcttccgtctgagcaagtctatatctaccaagggaatgtgccgggatcagtcgccagtgaagacattcagtcaccatatggaaaagtccccatgacctttaaacacgagctgttaaatcaacccccaattcaaatgccaggggggagtgtacgaattgtggattcttctaacttcccaatttcaaaaacgatagccgc

SEQ ID NO: 17 - DNA, 1430 bp, Bacillus subtilis
  source 1..1430: mol_type = genomic DNA
aaatgcttcacgatgggatgcttcattatctccttacattttgaccgcaaaagaccttgatagtccatcaaaccgcctccctactaaattaaatgaaggaaagcaaaaaaaagtaaactactactttctgactagcgcttgtgcaaatgctccggctgcagtttacgttttcttggatttccttcacttacatacatgactgccaaaaaagaagggaggtattttccatggatcaggtttttatagaggaagtcgtaaaacagatcggtaatctggggtttcctgcgctgattgcaatgtacctgctgacccgatttgagaaaaagtttgatcagctgatagaactgatgacagagctgaaagaccagaaagcaaaaaattaattttcaatcgaagttgactttttactgttttttttcacttaacaagacagaagggaaaacgaaaggcctttcaccttctctttctgcttaacatttatttttaaggaggagacagttacacttctcaaagatcccatttaaaaattttttttaaaaaaatatttgacatttttaaataaagcgtttataatatatgtagaaacaacaaagggggagatttgtttgataaggaggacaaacatgaaaaaacaaaacgacattccgcagccaatcagaggagacaaaggagccacggtgaaaatcccgcgcaatattgaaagagaccggcaaaatcctgatatgctcgttccgccggaaaccgatcatggaaccgtcagcaatatgaaattttcgttctctgatactcataaccggttagaaaaaggcggatatgcccgtgaagtcaccgtacgggagctgccgatttcagagaaccttgcatctgtaaatatgcggctgaagccaggtgccattcgcgagctgcattggcataaagaagcagaatgggcttatatgatttacggaagtgctagagtcacaattgtggatgaaaaagggcgcagctttattgatgatgtaggtgaaggagatctttggtacttcccgtcaggcctgccgcactccattcaagcgctggatgaaggagccgagttcctgctcgtgtttgacgatggatcattctctgaaaacagcacgttccagctgacagactggcttgcccacacccctaaagaagtcattgctgcgaactttggcgtgacaaaagaagaaatcgccaatctgccgggtaaagaaaaatatatatttgaaaaccaaatcccaggcagcttaaaagatgacattgtggaagggccgaacggcgaagtgccttacccgtttacttaccgccttcttgaacaggagccgattgaatcagagggaggaaaagtatacattgcggattcgacaaactttacagtgtctaaaacaatcgcatctgccctcgtgacggtagaactaaatagatag

SEQ ID NO: 18 - DNA, 1721 bp, Bacillus subtilis
  source 1..1721: mol_type = genomic DNA
gccgctatgttttttgaaatcgggaagttagaagagtccacaattcgtacgctccccccaggcgtttgaatgggggtttgatttaataactcgtatttgaaagtcaaggggacttttccgtaaggtgactgaatgtcttcactggcgactgatcccggcacatttccttgataaatatagacttgttctgaaggaagagagtgaaaagcattctctgggacaccaaaatttccagacagaacatcttttggcgtgtgtgcaagccaatctgaaatggttaatgttgaaaactcagaaaagtttccatcatcgaagacaagcagaaattcgcaatgttctaatccttgaatggaatgcggaattcctgcgggaaagtaccaaagatcgcctggaccaacatcagcaatgaaatttcgcccctcttggtcaactgcggtgatgcgggcccgtcccaaaagcatataagcccattccgcttgtttatgccaatgaagttcacgtacgcctcccgcagttaagctcatatttactcctgcaattgcggtcgaaatcggaagctgcctgacggtgatttcacgtgaccagccgccgtgatctaatttcatcggagcatctgagaatgaaaatctcaagttcggaatcataccttcgtctgtaacaggcggaacaagtatatttggattttgaatatctcgcatcatattacgcgggccagcatcaatagccccggctccatcacttcgaataggctgcggcacgtttctgtcttcatgattgattggttgtttttccaacagcatgtttgtcctccttatcaaacaaatctccccctttgttgtttctacatatattataaacgctttatttaaaaatgtcaaatatttttttaaaaaaaatttttaaatgggatctttgagaagtgcacctcgttttagaaattaacaagattttattccagcctatgcatgatcttatatggctgtgcctagaattcagcaaataagctatcttttataccatggattcttatgtaatttgatttcttctgtctgttctttccggcctgtcttccactctactttcactaagacatcatgtcctatcacttcatcttcaggcatataattaaaggtagcgatatggtcctccatgtgtcctccaccttgcatttcttcaccagtgtattctgataagtcgtactcatctatgttatcatcttgatactttttgccatctacatacagagtaattctttcaacaacagtccttttctcgtctttcttacttccttgccaatacaaatttagaaagtaattaggtccaattgacgtcttgtctgatttttcaaggttagaaacccacagtccgcttcttccgctgccttcatatttcactttgtttcctgagatcattacaatgattgctacgatgcagattattaagaatgcagcaatcatcaatagccattttttaatgttgttcttttttctcatgattttcctcacttccctcatacttagtaggaaaatggggtcaaaatgttactgataagaacttaaattaaagcgttataggggcaagattaaaacttataaacatgctcttacatgcattttttatttcattgagtcaattaccttcaaaatgattacattctttaacaaaatcccctatgattgtggtaatatctttgtgagatatttttgaattttgagaaagag